(19) United States
(12) Patent Application Publication
(10) Pub. No.: US 2021/0236594 A1
(43) Pub. Date: Aug. 5, 2021

(54) METHODS FOR IMPROVING FRAILTY AND AGING

(71) Applicant: Genome Protection, Inc., Buffalo, NY (US)

(72) Inventor: Olga CHERNOVA, Buffalo, NY (US)

(21) Appl. No.: 17/049,720

(22) PCT Filed: Apr. 24, 2019
(86) PCT No.: PCT/US19/28911

§ 371 Date: Oct. 22, 2020

Related U.S. Application Data
(60) Provisional application No. 62/662,028, filed on Apr. 24, 2018.

(51) Int. Cl. A61K 38/16; A61P 39/00

(52) U.S. Cl. CPC A61K 38/164; A61P 39/00

(57) ABSTRACT

In various aspects and embodiments provided are methods for treating age-related diseases or disorders and/or treating or preventing frailty in a patient. In certain embodiments the methods include administering a recombinant TLR5 agonist (e.g., a flagellin-based agent).

### CROSS-REFERENCE TO RELATED APPLICATIONS

This application claims the benefit of U.S. Provisional Application No. 62/662,028, filed Apr. 24, 2018, the entire contents of which are hereby incorporated by reference in their entirety.

### FIELD OF THE INVENTION

The present disclosure relates to compositions and methods for treating age-related diseases and/or improving frailty.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (Filename: “GPI-002PC_ST25.txt”; Date created: Apr. 24, 2019; File size: 59.9 KB).

### BACKGROUND

Aging of mammals is associated with accumulation of DNA damage in somatic cells, an increase in chronic systemic inflammation and reduced effectiveness of the immune system in clearing damaged cells.

TLR5 agonists derived from flagellin have been developed as therapies for various diseases. For example, entolimod is a pharmacologically-useful derivative of the natural TLR5 agonist flagellin currently being developed as a medical radiation countermeasure. In addition to its radioprotective activity, entolimod has demonstrated immunotherapeutic activity in preclinical cancer models.

There are currently no drugs or treatments that are conventionally used in medicine for prophylaxis and treatment of aging. Extension of healthy life and longevity has been documented by caloric restriction. A similar effect can be reached using mTOR inhibitors such as rapamycin. However, both require long-term applications. As such, pharmacological agents capable of slowing down the process of advancement of age-related frailty—both naturally occurring and accelerated by, e.g., cancer treatment—are needed.

### SUMMARY OF THE INVENTION

Accordingly, the present invention provides, in certain aspects, methods of improving or reducing and/or treating or preventing frailty in a patient, where the method includes: identifying a patient desiring or in need of frailty treatment or prevention, and administering to said patient a recombinant TLR5 agonist, where the recombinant TLR5 agonist is not fused to a pathogenic protein antigen.

In some embodiments, the frailty is age-related. In some embodiments, frailty comprises an accumulation of deficiencies in major physiological functions, reduction of regeneration capabilities, impaired wound healing and increased risk of age-related diseases. For example, in some embodiments, frailty is associated with natural aging or accelerated aging. Frailty can be measured according to any number of indices or tests known to one of skill in the art. For example, one such index, the Physiological Frailty Index (PFI), includes measurement of one or more parameters selected from grip strength, systolic blood pressure, diastolic blood pressure, blood flow volume, number of blood neutrophils, percentage of blood neutrophils, number of blood monocytes, percentage of blood monocytes, number of lymphocytes, number of red blood cells, hemoglobin levels, hematocrit levels, mean corpuscular volume, mean corpuscular hemoglobin levels, mean corpuscular hemoglobin concentration and keratinocyte-derived cytokine levels. Deviation from a reference standard in any one individual is known as a deficit, and the overall average PFI score of the individual is a ratio of deficits to the total number of parameters measured.

In some embodiments, the present invention provides methods of improving or reducing and/or treating or preventing frailty in a patient, as measured by a reduction in the PFI score of the patient. In some embodiments, methods and compositions of the present invention for improving or reducing and/or treating or preventing frailty in a patient include maintaining a PFI score over time so that the score increases at a rate slower than if the patient were not being administered the TLR5 agonist of the invention. In some embodiments of the present invention, the PFI score of the patient remains nearly the same over time. In further embodiments, methods of the present invention provide for a reduction in cellular senescence and immunosenescence associated with natural aging and/or accelerated aging (e.g., accelerated aging induced by, e.g., cancer or a cancer treatment).

In another aspect, the present invention provides for methods of treating or preventing an age-related disease or disorder in a patient, where the method includes: identifying a patient desiring or in need of treatment or prevention of an age-related disease or disorder, and administering to said patient a recombinant TLR5 agonist, where the recombinant TLR5 agonist is not fused to a pathogenic protein antigen. In some embodiments, the age-related disease or disorder is characterized by increased cellular senescence or immunosenescence.

In some embodiments, an age-related disease or disorder is selected from accelerated aging, cardiovascular disease, cerebrovascular disease, peripheral vascular disease, cardiac diastolic dysfunction, benign prostatic hypertrophy, aortic aneurysm, emphysema, atherosclerosis, diabetes, pulmonary fibrosis, blindness, dementia, Alzheimer's disease, kidney dysfunction, osteoarthritis, low grade chronic sterile inflammation, herniated intervertebral disc, frailty, hair loss, hearing loss, vision loss, muscle fatigue, skin conditions, skin nevi, wrinkly skin, hyperpigmentation, scarring, keloid, rosacea, vitiligo, ichthyosis vulgaris, dermatomyositis, actinic keratosis, and sarcopenia.

In specific embodiments, methods of the present invention include treating or preventing accelerated aging. In some embodiments, accelerated aging is a Progeroid syndrome or symptom thereof, including, but not limited to, Hutchinson-Gilford progeria syndrome (HGPS), Werner syndrome (WS), Bloom syndrome (BS), Rothmund-Thomson syndrome (RTS), Cockayne syndrome (CS), xeroderma pigmentosum (XP), trichothiodystrophy (TTD), combined xeroderma pigmentosum-Cockayne syndrome (XP-CS), or restrictive dermopathy (RD). Subjects having one of these diseases or disorders typically have reduced longevity (i.e., lifespan).

In further embodiments, accelerated aging is induced by a cancer or a cancer treatment. For example, it is contemplated by the invention that a cancer treatment that induces an acceleration in the natural aging process is selected from one or more therapies consisting of radiotherapy, hormonal, tyrosine kinase inhibitor, anthracycline, alkylating agent, topoisomerase inhibitor, antimetabolites/cytotoxic drug, BRAF inhibitor, antitumor antibiotic, isoquinololine alkaloid, Bcl-2 inhibitor, hematopoietic cell transplantation (HCT), telomerase inhibitor, nucleoside analogue reverse-transcriptase inhibitor, DNA cross-linking agent, ribonucleotide reductase inhibitor, microtubule inhibitor, and miRNA.

In some embodiments, any cancer is contemplated for which the patient receives treatment that can induce accelerated aging. In an embodiment, the cancer for which a patient receives treatment is hematological cancer. Further, in some embodiments, the patient received the cancer treatment during childhood.

In further embodiments, the recombinant TLR5 agonist is administered to the patient for at least one week, or at least one month, or at least six months, or at least one year, or at least two years, or at least three years, or at least four years, or at least five years after the patient received the cancer treatment. In some embodiments, the patient no longer has cancer or the patient is in remission at the time the recombinant TLR5 agonist is administered.

### BRIEF DESCRIPTION OF THE DRAWINGS

The foregoing features of embodiments will be more readily understood by reference to the following detailed description, taken with reference to the accompanying drawings, in which:

FIG. 1A-B. Longevity and chronological aging of NIH Swiss male and female mice. A. Kaplan-Meier survival curves for male and female NIH Swiss mice (n=79/group). No statistically significant difference between the two sexes was detected with mean lifespan 89.13+3.49 and 96.95+2.99 weeks for males and females respectively (p=0.368, log-rank test). B. Physiological Frailty Index (PFI) created for chronologically aged male and female NIH Swiss mice of different ages. Age-dependent increase in PFI reflects accumulation of health deficits observed in mice of both sexes.

FIG. 2. Schematic diagram of experiment schedule for flagellin treatment. In each experimental group males and females received a short course of Flagellin injected s.c. daily (1 mg/injection) for five consecutive days: group 1 at the age of 55 weeks, group 2 at 44^{th} week, and group 3 received two courses at 18^{th} and 84^{th} weeks. PFI was evaluated when mice reach 26, 52, 78, 104, and 120 weeks.

FIG. 3. Single five days course of flagellin administration at the age of 55 weeks (experimental group 1) increased mean survival of NIH Swiss male mice from 89±7.9 to 112.3±9.2 weeks. No effect on female mice survival rate was detected. Physiological Frailty Index was created for male and female mice at 104 and 120 weeks of age. Five days injection course of flagellin earlier in life significantly reduced PFI of male but not female mice.

FIG. 4. Dynamic changes in Physiological Frailty Index in mice from experimental group 1. At 104 weeks, the average PFI in flagellin treated male group (dashed line) increased by 33% from the time of treatment at 55 weeks, while in control mice (solid line) it steadily increased by 100%. No effect was observed in the female group.

FIG. 5. Dynamic changes in Physiological Frailty Index in mice from experimental group 2. Mice were injected with flagellin at the age of 44 weeks. The average PFI evaluated at 78 and 104 weeks demonstrated almost no changes in flagellin treated male group (dashed line) from the time of treatment, while in control mice it steadily increased more then 200% (solid line). No effect was observed in the female group.

FIG. 6. Dynamic changes in Physiological Frailty Index in mice from experimental group 3. Mice were injected with flagellin twice at the age of 18 and 84 weeks. The average PFI evaluated four times at the age of 52, 78, 88, and 104 weeks demonstrated significantly smaller increase in flagellin treated male group (dashed line) as compared to control mice (solid line). No effect was observed in the female group.

FIG. 7. Chronic administration of Rapatar (nanoformulated water soluble rapamycin, see Comas, et al., Aging (Albany N.Y.) 10:715-22 (2012)) extends lifespan of female, but not male NIH Swiss mice. Kaplan-Meier survival curves of mice that start receiving Rapatar in drinking water at 89 weeks of age. Chronic administration of rapamycin increases lifespan of female mice from 114.4±3.1 to 127.3±3.1 weeks (p=0.01, Kaplan-Meier log-rank test).

FIG. 8. Schematic illustration of the timeline (not to scale) for treatment and evaluation of NIH Swiss mice. “Entolimod” arrows indicate timing of entolimod treatment (or PBS treatment in control groups) and “PFI” arrows indicate timing of PFI determination.

FIG. 9. Effect of entolimod treatment on longevity of “old” mice. Kaplan-Meier survival curves are shown for groups of male and female NIH Swiss mice that received 5 daily SQ injections of entolimod (5 μg/mouse; dark line) or PBS (light line) at 113 weeks of age (arrow).

FIG. 10. Effect of entolimod treatment on longevity of “middle-aged” mice. Kaplan-Meier survival curves are shown for groups of male and female NIH Swiss mice that received 5 daily SQ injections of entolimod (5 μg/mouse; dark line) or PBS (light line) at 55 weeks of age (arrow).

FIG. 11. Effect of entolimod treatment on longevity of “young” mice. Kaplan-Meier survival curves are shown for groups of male and female NIH Swiss mice that received 5 daily SQ injections of entolimod (5 μg/mouse; dark line) or PBS (light line) at 18 weeks of age and at 84 weeks of age (arrow).

FIG. 12. Treatment of NIH Swiss mice with entolimod at 112 weeks of age had no effect on mean PFI measured at 128 weeks of age. The number of animals evaluated for each group is shown in white within the bars of the graph

FIG. 13. Treatment of NIH Swiss mice with entolimod at 55 weeks of age led to reduced PFI at 104 weeks and 120 weeks in males, but not females. The number of animals evaluated for each group is shown in white within the bars of the graph.

FIG. 14. Dynamics of changes in PFI in male and female NIH Swiss mice after receiving entolimod treatment at 18 and 84 weeks of age. Mean PFI was determined at 18, 52, 84 and 104 weeks of age for entolimod-treated (black circles) and PBS-treated (grey circles) groups of mice. Timing of treatments is indicated by arrows.

### DETAILED DESCRIPTION OF THE INVENTION

Some of the aspects and embodiments of this instant disclosure are based, at least in part, on the finding that TLR5 agonists (e.g., recombinant flagellin and/or flagellin-based agents, such as entolimod) that are not fused to a pathogenic protein antigen, can be effective, for example, in improving and/or treating or preventing frailty and/or treating or preventing age-related diseases or disorders, and/or preventing or slowing aging (including accelerated aging).

The aging process is manifested by a gradual accumulation of deficiencies in all major physiological functions, reduction of regeneration capabilities, impaired wound healing and increased risk of age-related diseases or disorders such as cancer, diabetes type 2, arthritis, Alzheimer and Parkinson diseases, atherosclerosis and others. Cumulatively, all these events can be described as a gradual increase in frailty and measured by a so-called “frailty index”. Age-related increase in frailty can be expedited in people or animals that underwent cancer treatment by chemotherapy and radiation, which can be interpreted as accelerated aging.

Without wishing to be bound by theory, the present invention contemplates that the progression of natural aging, as well as aging accelerated by, e.g., cancer treatment, can be dramatically slowed down by activation of natural innate immunity mechanism of response to infection with bacteria that have flagella—an organelle for active moving that is built with the protein named flagellin; presence of such bacteria in the body is recognized by a cell surface receptor named Toll-like receptor 5 (TLR5). Binding of a TLR5 agonist, e.g. a flagellin or flagellin-based agent (such as entolimod) to TLR5 triggers a physiological response leading to systemic mobilization of immune system accompanied with production of multiple bioactive factors (cytokines, chemokines, etc.) that have long-term effect on the organism manifested as a slowdown of frailty acquisition and improved health and quality of life of the treated organisms. Treatment with flagellin or its derivatives capable of activation of TLR5 can be projected as an approach to prevent and treat natural aging and premature accelerated aging caused by cancer treatment and other types of poisoning.

Aging is a gradual systemic pathological transformation of mammalian organism advancing with time. It is associated with accumulation of multiple deficiencies in functions of multiple organs and tissues and reduced regeneration capabilities leading to development of age-related chronic diseases or disorders including atherosclerosis, diabetes, pulmonary fibrosis, blindness, dementia, kidney dysfunction, osteoarthritis, and low grade chronic sterile inflammation as well as other age-related diseases and disorders contemplated herein. These conditions frequently coincide with a gradual development of geriatric syndromes including frailty, cognitive impairment and immobility. Aging is a natural and unavoidable process. Underlying causes of aging are still disputable; however, two features of aging are generally accepted as universal: an increase in DNA damage and development of systemic sterile chronic inflammation, both considered as major contributors of age-related pathologies.

In various embodiments, the present invention provides methods of reducing aging, or the multiple deficiencies causes thereof. In various embodiments, the present invention provides methods of reducing the amount or cellular impact of DNA damage. In various embodiments, the present invention provides methods of reducing the amount or cellular impact of systemic sterile chronic inflammation.

In various embodiments, the present invention provides methods of improving the cellular clearance of damaged cells, e.g. that may be functionally declined in aged subjects

In various embodiments, the present invention relates to treating or preventing cellular senescence, for example by reducing, halting, or delaying the senescence. Without intending to be bound by any particular theory, cellular aging (senescence) is considered to be caused by overstimulation and overactivation of signal transduction pathways such as the mTOR pathway, especially when the cell cycle is blocked, leading to cellular hyperactivation and hyperfunction. In turn, this causes secondary signal resistance and compensatory incompetence. Both cellular hyperfunction and signal-resistance cause organ damage (including in distant organs), manifested as aging (subclinical damage) and age-related diseases or disorders (clinical damage), eventually leading to organismal death. Non-limiting example of markers of cellular aging include cellular hypertrophy, permanent loss of proliferative potential, large-flat cell morphology and beta-Gal staining. In various embodiments, the present invention relates to modulating any of the markers of cellular aging.

### Frailty and Frailty Indices

In various embodiments, the present invention provides methods of improving or reducing and/or treating or preventing frailty in a patient, wherein the method includes: identifying a patient desiring or in need of frailty treatment or prevention, and administering to said patient a recombinant TLR5 agonist, wherein the recombinant TLR5 agonist is not fused to a pathogenic protein antigen.

In some embodiments, frailty comprises an accumulation of deficiencies in major physiological functions, reduction of regeneration capabilities, impaired wound healing and increased risk of age-related diseases. For example, in some embodiments, frailty is associated with natural aging or accelerated aging. Frailty can be measured according to any number of indices or tests known to one of skill in the art. For example, one such index, the Physiological Frailty Index (PFI), includes measurement of one or more parameters selected from grip strength, systolic blood pressure, diastolic blood pressure, blood flow volume, number of blood neutrophils, percentage of blood neutrophils, number of blood monocytes, percentage of blood monocytes, number of lymphocytes, number of red blood cells, hemoglobin levels, hematocrit levels, mean corpuscular volume, mean corpuscular hemoglobin levels, mean corpuscular hemoglobin concentration and keratinocyte-derived cytokine levels. Deviation from a reference standard in any one individual is known as a deficit, and the overall average PFI score of the individual is a ratio of deficits to the total number of parameters measured.

Frailty can manifest as vulnerability to stressors and a reduced capacity to withstand stress. For example, the disclosure of Buchner and Wagner 1992 Clin Geriatr Med. 1992 February; 8(1):1-17 is hereby incorporated by reference in its entirety. Frailty can manifest as loss of complexity of homeostatic mechanisms (e.g., interconnectedness and/or feedback or feedforward). For example, the disclosure of Lipsitz 2002 J Gerontol A Biol Sci Med Sci. 2002 March; 57(3):B115-25. is hereby incorporated by reference in its entirety. Frailty can also manifest as disuse and/or a decrease in energy flow through an organism, as described in Bortz 2002, J Gerontol A Biol Sci Med Sci. 2002 May; 57(5):M283-8. which is hereby incorporated by reference in its entirety. Frailty can also manifest as homeostatic dysregulation, as described by Ferrucci 2005 J. Gerontol. A Biol. Sci. Med. Sci. 60, 56, which is hereby incorporated by reference in its entirety.

There are several comprehensive approaches for quantitative assessment of aging-related accumulation of deficits and frailty in humans and animals. Individual organisms are heterogeneous in their health status and the rate of aging. To account for such heterogeneity, a Frailty Index (FI) has been introduced as a numerical score which is a ratio of the deficits present in a person to the total number of deficits considered in the study. Changes in the FI characterize the rate of individual aging. A similar approach has been applied to laboratory animals. Frailty index is considered as a reliable and broadly accepted measure of “biological age” and the degree of general health decline indicative of a reduction in the quality of life.

In certain aspects and embodiments, provided herein includes methods for improving and/or treating or preventing frailty and/or reducing frailty index in a patient. Frailty can be assessed in any of many methods known in the art. For example, frailty and methods to evaluate/index frailty are described in Hubbard, et al., Ageing, published electronically November, 2008 page 115-118; Cesari, et al., Age and Ageing, 43:10-12, 2014; and Mohler et al., Experimental Gerontology, 54:6-13, 2014, all of which are hereby incorporated by reference.

In various embodiments, a Frailty Index is calculated as described in U.S. Patent Application Publication No. 2015/0285823, which is incorporated herein by reference. For example, a description of the determination of the Frailty Index is provided. The Frailty Index was developed to assess a fit to frail range for the organisms of the same chronological age to address the notion that chronological age does not always reflect biologic age. Based on sixteen-item parameters (that include measurements of weight, grip strength, blood pressure, complete blood count, cytokine level analysis), FI is calculated as a ratio of the total number of deficits measured and are assigned a score of FI between 0 (no deficits=fit) and 1 (all deficits present=frail). Therefore, higher FI indicates poorer health of an organism. In this regard, a FI is provided as a useful tool for assessing a “fit” to “frail” range organisms of the same chronological age.

In certain embodiments, methods of the present invention reduce or prevent frailty in a subject as measured according to the Physiological Frailty Index (PFI), as described in Antoch et al. Aging. 2017; 9: 1-12 (hereby incorporated by reference in its entirety). For example, PFI can be determined for an individual subject with reference to a young reference subject. For each subject, various parameters are measured. These parameters include non-invasive measurements, including age, body weight, grip strength, and diastolic blood pressure. Additional blood chemistry measurements may also be determined, including white blood cell count, neutrophil count, neutrophil percentage, lymphocyte percentage, monocyte percentage, eosinophil percentage, red blood cell count, hemoglobin levels, hematocrit levels, mean corpuscular volume, mean corpuscular hemoglobin levels, mean corpuscular hemoglobin concentration, platelet count, and mean platelet volume. For each parameter mean value and standard deviation are calculated. Subjects differing in more than one standard deviation (STDEV) from mean value in any single parameter are excluded from the reference group. The value for each parameter measured for subjects of older ages is compared with the corresponding value for the reference group and assigned a score. Values that differ less than 1 STDEV are assigned the score of 0 (no deficit, within the range of the reference group). Values that are different for one STDEV are scored as 0.25 (minimal deficit). Values that differ from the corresponding values in the reference group by 2 STDEV are scored as 0.5 and those that differ by 3 STDEV are scored as 0.75. If the value is above 3 STDEV, it is scored as 1 (extreme deficit). The number of deficits the individual subject expressed is calculated as a ratio of the total number of parameters measured and is referred to as Physiological Frailty Index (PFI).

In some embodiments, methods of the present invention reduce or improve and/or treat or prevent frailty in a subject, as measured by the PFI. For example, administering the recombinant TLR5 agonist to a subject in order to reduce or improve and/or treat or prevent frailty can result in a reduced PFI score. In some embodiments, a subject's PFI score is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In some embodiments, a subject's PFI score is reduced by about 25%-75%, about 25%-50%, or about 50% to 75%. In further embodiments, a subject's PFI score is reduced to no greater than 0.9, 0.85, 0.8, 0.75, 0.7, 0.65, 0.6, 0.55, 0.5, 0.45, 0.4, 0.35, 0.3, 0.25, 0.2, 0.15, 0.1 or 0.5.

Further, frailty as an accumulation of deficits can be measured by the Rockwood frailty index, as described in Rockwood et al., J Gerontol A Biol Sci Med Sci. 2007 July; 62(7):722-727, which is incorporated by reference in its entirety. In embodiments, the present methods reduce or prevent frailty as assessed by the Rockwood frailty index.

Frailty as a biologic syndrome of decreased reserve resulting from cumulative declines across multiple physiologic systems can be measured by the Fried frailty score, as described in Fried et al., J Gerontol A Biol Sci Med Sci. 2001 March; 56(3):M146-56, which is incorporated by reference in its entirety. The Fried frailty score comprises a Physical Frailty Phenotype (PFP), which measures various parameters, such as weight loss of more than 10 pounds; weakness as related to grip strength; self-reported exhaustion; 15 feet walking speed; and amount of physical activity in Kcals per week. The Fried frailty score incorporates scoring of 0 (not frail), 1-2 (intermediate frailty), and greater than or equal to 3 (frail). In various embodiments, methods of the present invention reduce or improve and/or treat or prevent frailty in a subject, as measured by a Fried frailty score. For example, administering the recombinant TLR5 agonist to a subject in order to reduce or improve and/or treat or prevent frailty can result in a reduced Fried frailty score from 3 to 2, from 3 to 1, from 3 to 0, from 2 to 1, from 2 to 0 or from 1 to 0. Further, in some embodiments, administering the recombinant TLR5 agonist to a subject in order to reduce or improve and/or treat or prevent frailty results in a lack of increase of a subject's Fried frailty score.

Frailty can also be measured by the FRAIL Scale, as described in Abellean Van Kan et al., J Am Med Dir Assoc. 2008 February; 9(2):71-2. doi: 10.1016/j.jamda.2007.11.005, which is incorporated by reference in its entirety. The parameters measured in the FRAIL Scale include feelings of persistent fatigue; resistance (ability to climb a single flight of stairs); ambulation (ability to walk one block); more than five illnesses; and more than 5% loss of weight. The FRAIL Scale incorporates scoring of 0 (not frail), 1-2 (intermediate frailty), and greater than or equal to 3 (frail). In various embodiments, methods of the present invention reduce or improve frailty in a subject, as measured by a FRAIL Scale score. For example, administering the recombinant TLR5 agonist to a subject in order to reduce or improve frailty can result in a reduced FRAIL Scale score from 3 to 2, from 3 to 1, from 3 to 0, from 2 to 1, from 2 to 0 or from 1 to 0. Further, in some embodiments, administering the recombinant TLR5 agonist to a subject in order to reduce or improve and/or treat or prevent frailty results in a lack of increase of a subject's FRAIL Scale score.

In some embodiments the methods as provided herein improve (or reduce) frailty index, or delay or slow a decline in frailty using at least one accepted measure of fraility. In some embodiments the methods as provided herein improve (or reduce) frailty index, or delay or slow a decline in frailty using at least one accepted measure of fraility selected from the Frailty Index (FI), the Physiological Frailty Index (PFI), Fried frailty score, Rockwood frailty index, FRAIL Scale and the modified frailty index.

In some embodiments, the frailty comprises low lean mass, weakness, exhaustion, low energy expenditure and/or slow walking speed. In embodiments, the present methods reduce or prevent the onset or development of one or more of low lean mass, weakness, exhaustion, low energy expenditure and/or slow walking speed.

### Age-Related Diseases and Disorders

The present invention contemplates methods involving administering a recombinant TLR5 agonist that is not fused to a pathogenic protein antigen. In various embodiments, the recombinant TLR5 agonist decreases cellular senescence in the patient having an age-related disease or disorder.

In some embodiments, the disease is cancer, age-related disease, tobacco-related disease, or skin wrinkles.

For example, in some embodiments, the methods provided herein are to prevent or treat age-related diseases or disorders such as Alzheimer's disease, type II diabetes, macular degeneration, chronic inflammation-based pathologies (e.g., arthritis), and/or to prevent development of cancer types known to be associated with aging (e.g., prostate cancer, melanoma, lung cancer, colon cancer, etc.), and/or with the purpose to restore function and morphology of aging tissues (e.g., skin or prostate), and/or with the purpose to improve morphology of tissue impaired by accumulated senescent cells (e.g., cosmetic treatment of pigmented skin lesions), and/or with the purpose to improve the outcome of cancer treatment by radiation or chemotherapy, and/or with the purpose to prevent recurrent and metastatic disease in cancer patients by elimination of dormant cancer cells. The disclosure is suitable for prophylaxis and/or therapy of human and non-human animal diseases and aging and age-related disorders.

In various examples, the disclosure relates to methods of treating an individual suspected of having or at risk for developing an age-related disease or disorder, including but not necessarily limited to Alzheimer's disease, Type II diabetes, macular degeneration, or a disease comprising chronic inflammation, including but not necessarily limited to arthritis.

In some embodiments, the methods described herein or for treatment of a patient identified as having or at risk of having a cardiovascular disease or disorder, inflammatory disease or disorder, pulmonary disease or disorder, neurological disease or disorder, metabolic disease or disorder, dermatological disease or disorder, age-related disease or disorder, a premature aging disease or disorder, and a sleep disorder.

The methods provided herein in certain aspects and embodiments are applicable to treating or preventing degenerative disorders that accompany aging. More particularly, the methods provided herein may provide improvements in 1) reducing the rate at which adipose tissue is lost, 2) reducing the rate at which muscle fibre diameter is reduced, and 3) reducing the rate at which skin tone deteriorates over time. These effects are likely to be seen more dramatically in aged recipients, i.e. those at an age greater than 50 years, especially those aged greater than 60 years or more, such as 65 years, 70 years and 75 years and greater. Also, candidate recipients include those whose lifestyle imposes age-accelerating effects, including tobacco smokers and users, alcohol and narcotic drug abusers, skin tanning enthusiasts, and the like.

Particular conditions and diseases or disorders that are treated by the present methods, in various embodiments, include sarcopenia. Sarcopenia is characterized first by a muscle atrophy (a decrease in the size of the muscle), along with a reduction in muscle tissue “quality,” caused by such factors as replacement of muscle fibres with fat, an increase in fibrosis, changes in muscle metabolism, oxidative stress, and degeneration of the neuromuscular junction. Combined, these changes lead to progressive loss of muscle function and frailty.

Other conditions that are treated by the present method, in various embodiments, include cataracts, and so-called “signs of aging” such as wrinkling and discoloration of the skin, and overall dermal tone. Treatment by the present method is expected to reduce the rate at which fat and muscle that support skin tone are reduced, so that skin wrinkling also is reduced, delayed or eliminated. As well treatment is expected to have a benefit on the rate at which cataracts form in the eye.

### Accelerated Aging (Cancer and Progeroid Syndrome)

In some embodiments, the present invention provides methods for reducing accelerated aging in a subject. For instance, in some embodiments, the present invention relates to the administration of a recombinant TLR5 agonist, e.g. flagellin or flagellin-based agent (such as entolimod) to a subject or patient to reduce accelerated aging associated with cancer and/or cancer treatments or Progeroid syndromes.

Exposure of younger individuals to genotoxic medical treatments or environment has been linked to a high risk of premature development of multiple aging-associated conditions listed above and considered as accelerated aging.

One of the most common medical treatments of this type is cancer treatment. Cancer treatment frequently involves exposure of humans and animals to genotoxic stresses leaving numerous normal cells with damaged DNA, provoking accumulation of senescent cells and acquisition of chronic systemic inflammation. These conditions increase the risk of multiple diseases or disorders commonly associated with natural aging such as abnormal thyroid function, decreased bone mineral density and increased osteoporosis, infertility, compromised tissue regeneration, cardiotoxicity, pulmonary fibrosis and chronic sterile inflammation.

In various embodiments, the cancer being treated is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

Acceleration of aging in cancer survivors is especially well documented in individuals that were successfully treated for cancer in their childhood. In fact, adults treated for childhood cancer are at increased risk of early development of chronic health conditions such as cardiovascular, pulmonary, hepatic, renal, and gonadal dysfunction, and secondary malignant neoplasms and increased rate of mortality. The rates of chronic diseases among survivors in their 20s are similar to rates among siblings in their 50s. Elevated rates of other aging-associated conditions, such as cognitive dysfunction, and reduced muscle strength, are also reported among childhood cancer survivors and appear decades earlier than expected. This and other studies suggest that some survivors of childhood cancer have a physiological frailty phenotype consistent with that found among older adults. Physiologic frailty among hematopoietic cell transplantation (HCT) survivors also suggests accelerated aging and is a predictor for premature mortality. Rates of frailty were eightfold higher among HCT survivors than among their siblings. Among survivors of HCT at least 10 years after transplant, the 15-year cumulative incidence of severe/life-threatening/fatal conditions was 41%.

In various embodiments, methods of the present invention include treating or preventing premature or accelerated aging. In some embodiments, accelerated aging is a symptom of any one of the Progeroid syndromes, including, but not limited to, Hutchinson-Gilford progeria syndrome (HGPS), Werner syndrome (WS), Bloom syndrome (BS), Rothmund-Thomson syndrome (RTS), Cockayne syndrome (CS), xeroderma pigmentosum (XP), trichothiodystrophy (TTD), combined xeroderma pigmentosum-Cockayne syndrome (XP-CS), or restrictive dermopathy (RD). Subjects having one of these diseases or disorders typically has reduced longevity (i.e., lifespan).

In various embodiments, the methods of the present invention modulate (e.g., increase or decrease) levels of inflammation in a subject. “Inflammation” is a normal response to a variety of acute stresses on the body, including infection, fever and injury. Other types of inflammation include increased levels of pro-inflammatory cytokines found within tissues and systemically in plasma. Inflammation may be associated with infections, but it occurs in response to virtually any type of injury or threat, including physical trauma, cold, burns from radiation, heat or corrosive materials, chemical irritants, bacterial or viral pathogens, localized oxygen deprivation (ischemia) or reperfusion (sudden reinfusion of oxygen to ischemic tissue), and others. It includes the classic symptoms of redness, heat, swelling, and pain, and may be accompanied by decreased function of the inflamed organ or tissue. It is a generalized reaction involving several effects that may tend to combat an injurious agent that may be present at the site where an injury or threat was detected, or it may tend to contain the injury or threat to its initial location, to keep it from spreading rapidly. Inflammation is a self-defensive reaction aimed at eliminating or neutralizing injurious stimuli, and restoring tissue integrity. Like peripheral inflammation, neuroinflammation can become a harmful process, and it is now widely accepted that it may contributes to the pathogenesis of many central nervous system disorders. CNS inflammation is commonly associated with some degree of tissue damage including, loss of myelin sheaths or loss of axons, and is a central theme in human patients with MS. The level of inflammation can be quantified by performing a simple blood test for a particular compound called C-reactive protein, or CRP.

In various embodiments, the methods of the present invention decrease levels of sterile chronic systemic inflammation in a subject. “Sterile chronic systemic inflammation”, named “inflammaging” is a characteristic of aging. Chronic inflammation causes damage over time to organ systems like the heart, brain and kidneys, leading to disability or premature death. Blood vessels that supply these organs are vulnerable to inflammation, leading to vessel wall-thickening and narrowing of the blood passageway. Elevated CRP levels, measured over time, are an indicator of chronic inflammation in humans. Studies have shown that elevated levels of CRP correlate with an increased risk of heart attack and stroke. Aging is an intricate process that results from a combination of environmental, genetic, epigenetic, and stochastic factors. A chronic proinflammatory status is a pervasive feature of aging. This chronic, low-grade, systemic inflammation occurring in the absence of overt infection (sterile inflammation) has been defined as “inflammaging” and represents a significant risk factor for morbidity and mortality in the elderly. Prattichizzo et al in (Inflammaging” as a Druggable Target: A Senescence-Associated Secretory Phenotype-Centered View of Type 2 Diabetes) Oxid Med Cell Longev. 2016 and Nasi et al in (Aging and inflammation in patients with HIV infection), Clin Exp Immunol. 2016 May 20, explore the connection between aging and inflammation.

In various embodiments, methods of the present invention treat or prevent age-related diseases or disorders in a subject. The term “age-related disease or disorder” includes but is not limited to a disease or disorder in an adult such as cancer, a metabolic disease, cardiovascular disease, tobacco-related disease, or skin wrinkles. Cancer includes but is not limited to prostate cancer, colon cancer, lung cancer, squamous cell cancer of the head and neck, esophageal cancer, hepatocellular carcinoma, gastric cancer, pancreatic cancer, ovarian cancer, or breast cancer. Age-related or tobacco-related disease or disorder includes cardiovascular disease, cerebrovascular disease, peripheral vascular disease, Alzheimer's disease, osteoarthritis, cardiac diastolic dysfunction, benign prostatic hypertrophy, aortic aneurysm, or emphysema.

In various embodiments, methods of the present invention mediate rejuvenation in a subject. The term “rejuvenation” refers to the results of reducing or preventing the progress of aging and/or reducing or preventing the progress of an age-related disease or disorder. The term “rejuvenating” refers to a process of improving parameters of frailty index and/or other markers of aging cell phenotypes or markers of age-related disease or disorder states, e.g., improved muscle endurance or strength, improved glucose tolerance, decreased presence of systemic or local inflammatory cytokines, improved mitochondrial function, and erasing epigenetic modifications participating in the cellular aging phenotype. In some embodiments, the loss or reduction of the expression at least one of the markers identified as having increased expression in adipose tissue macrophages (ATMs) from aged mice (Garg, S. K. et al. Crit Rev Immunol. 2014; 34(1). 1-14): CD11c, CD206, Mgl1, IL-6, TNF-alpha, Nos2, Ccr-7, IL-12, Arg1, Ccl-2, Ccr-1, Ccr-5, Ccr-9, Mcp-1, Cxcr-3, IL-1beta may also be considered a sign of rejuvenation.

### Lifespan

In some embodiments, the present invention provides methods for increasing a subject's longevity or lifespan. For instance, in some embodiments, the present invention relates to the administration of a recombinant TLR5 agonist, e.g. flagellin or flagellin-based agent (such as entolimod) to a patient to increase longevity or lifespan.

For example, the present invention may increase a subject's longevity or lifespan by at least about 5, at least about 10, at least about 15, at least about 20, or at least about 25 years, as compared to a subject that is not administered the recombinant TLR5 agonist described herein and/or as compared to a life expectancy calculation, as described herein. Further, various embodiments of the present invention contemplate methods that reduce or decrease cellular senescence and/or immunosenescence in a subject.

In various embodiments, an increase in longevity or lifespan is assessed relative to a comparable population. For example, an increase in longevity or lifespan is assessed relative to a cohort—e.g. cohort LEB, the mean length of life of an actual birth cohort (all individuals born a given year) or a period—e.g. period LEB, the mean length of life of a hypothetical cohort assumed to be exposed, from birth through death, to the mortality rates observed at a given year. Such assessments can be made relative to various reports on lifespan and/or longevity in the art (e.g. World Health Organization (WHO)'s Health Status Statistics: Mortality). In some embodiments, the present methods provide for increased longevity or lifespan than what is expected relative to comparable populations. In some embodiments, the present methods provide for increased longevity or lifespan than what is expected relative to various reports on lifespan and/or longevity in the art (e.g. World Health Organization (WHO)'s Health Status Statistics: Mortality).

In further embodiments, an increase in longevity or lifespan is assessed with reference to one or more actuarial life tables, e.g. Life Tables For The United States Social Security Area 1900-2100 (Actuarial Study No. 120, Bell and Miller). In some embodiments, the present methods provide for increased longevity or lifespan than what is expected relative to one or more actuarial life tables.

### Subjects

The methods provided herein can be used with a patient that is a mammal, including humans and non-human mammals. Non-human mammals treated using the present methods include domesticated animals (i.e., canine, feline, murine, rodentia, and lagomorpha) and agricultural animals (bovine, equine, ovine, porcine). In various examples, the individual to whom a compound or composition is administered is an individual who is at risk for, is suspected of having or has been diagnosed with an age-related disease or disorder.

In various embodiments of the present invention, the patient is a young human, a middle-aged human, or an elderly human. For example, in some embodiments, the patient is between about 18 and about 35 years, or between about 18 and about 30 years, or between about 18 and about 25 years, or between about 18 and about 20 years. In some embodiments, the patient is between about 36 and about 55 years, or between about 40 and about 55 years, or between about 45 and about 55 years, or between about 36 and about 50 years, or between about 36 and about 45 years, or between about 36 and about 40 years, or between about 40 and about 50 years old, or between about 45 and about 55 years old. In some embodiments, the patient is between about 56 and about 85 years, or between about 60 and about 85 years, or about 65 and about 85 years, or between about 70 and about 85 years, or between about 75 and about 85 years, or between 80 and about 85 years, or between 56 and about 80 years, or between 56 and about 75 years, or between 56 and about 70 years, or between 56 and about 65 years, or between 56 and about 60 years, or between about 60 years and about 80 years, or about 65 years and about 75 years.

In some embodiments, the patient is about 1, or about 2, or about 3, or about 4, or about 5, or about 6, or about 7, or about 8, or about 9, or about 10, or about 11, or about 12, or about 13, or about 14, or about 15, or about 16, or about 17, or about 18, or about 19, or about 20, or about 21, or about 22, or about 23, or about 24, or about 25, or about 26, or about 27, or about 28, or about 29, or about 30, or about 31, or about 32, or about 33, or about 34, or about 35, or about 36, or about 37, or about 38, or about 39, or about 40, or about 41, or about 42, or about 43, or about 44, or about 45, or about 46, or about 47, or about 48, or about 49, or about 50, or about 51, or about 52, or about 53, or about 54, or about 55, or about 56, or about 57, or about 58, or about 59, or about 60, or about 61, or about 62, or about 63, or about 64, or or about 65, or about 66, or about 67, or or about 68, or about 69, or about 70, or about 71, or about 72, or about 73, or about 74, or about 75, or about 76, or about 77, or about 78, or about 79, or about 80, or about 81, or about 82, or about 83, or about 84, or about 85 years old. In some embodiments, the patient is at least 55 years old.

A person of skill in the art will contemplate that age ranges with respect to “young,” “middle-aged,” and “elderly” definitions can vary based on geographic region, among other factors. Petry, Gerontologist 2002 February; 42(1):92-9 describes age-related definitions and is hereby incorporated by reference in its entirety.

In embodiments, the biological sex of the patient is male or female. In embodiments, the biological sex of the patient is male. In embodiments, the biological sex of the patient is female.

In embodiments, the biological sex of the patient is male and the patient is middle aged (e.g. between about 36 and about 55 years, or between about 40 and about 55 years, or between about 45 and about 55 years, or between about 36 and about 50 years, or between about 36 and about 45 years, or between about 36 and about 40 years, or between about 40 and about 50 years old, or between about 45 and about 55 years old). In some embodiments, the present methods, e.g. as applicable to a middle aged male patient, prevent or reduce the severity of one or more frailties and age-related diseases or disorders.

In various embodiments of the present invention, the subject is a patient. In some embodiments, the patient is a middle-aged human. For example, in some embodiments, the patient is between about 35 and 55 years old. In further embodiments, the biological sex of the patient is male.

In some embodiments of the methods provided herein, the patient is a mammal. In some embodiments of the methods provided herein, the patient is a human. In certain embodiments of the methods provided herein, the patient is a male.

### TLR5 Agonists and Derivatives Thereof

Toll-like receptors (TLRs) play a central role in the initiation of cellular innate immune responses. They recognize pathogen-associated molecular patterns (PAMPs) that are expressed on infectious agents and mediate the production of cytokines necessary for the development of effective immunity. There are 10 TLR genes in humans and 12 in mice. In particular, Toll-like receptor 5 (TLR5) is a transmembrane protein that recognizes bacterial flagellin and is highly expressed in the intestinal mucosa. Vertebrate organisms recognize the presence of potentially pathogenic flagella-carrying bacteria via signaling activated by a highly specific interaction of flagellin with TLR5 that triggers a cascade of signal transduction events aimed at activation and mobilization of natural defense mechanisms of innate immunity. Activation of TLR5 by entolimod (CBLB502), a pharmacologically-useful flagellin derivative, was capable of protecting animals from lethal total body irradiation.

As used herein, the term “TLR5 agonist” refers to a compound or peptide that selectively activates or increases normal signal transduction through TLR5. In some embodiments of the present invention, the TLR5 agonist is recombinant. In some embodiments, a TLR5 agonist has an EC50 of less than about 10⁻⁷M; or less than 10⁻⁸ M; or less than 10⁻⁹M; or less than 10⁻¹ M; or less than 10⁻¹° M; or less than 10⁻¹¹ M. In certain embodiments, a TLR5 agonist as provided herein has an EC50 of less than about 10⁻⁷M; or less than 10⁻⁸ M; or less than 10⁻⁹M; or less than 10⁻¹⁰ M; or less than 10⁻¹⁰ M; or less than 10⁻¹¹M in the flagellin bioactivity assay using HEK-Blue™-hTLR5 cells (Invivogen) as described in Lu Y., et al., Biotechnol. Bioeng. 110, 2073-2085 (2013) and in Lu and Swartz, Sci Rep 6:18379 (2016) or a similar TLR5 bioactivity assay.

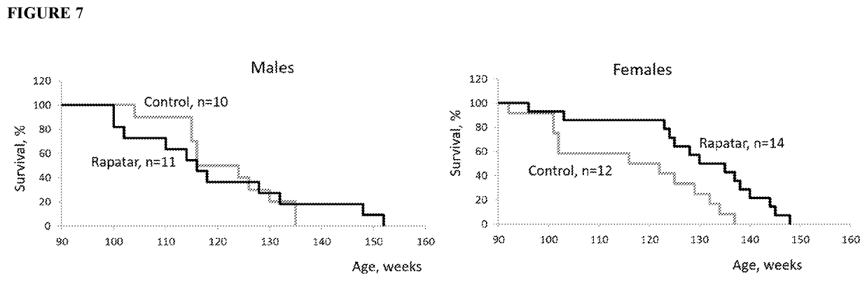

In some embodiments, a TLR5 agonist that is not fused to a pathogenic protein as provided herein is a flagellin-based agent. As used herein, the term “flagellin” means flagellin polypeptide contained in a variety of Gram-positive or Gram-negative bacterial species. The nucleotide and amino acid sequences of flagellin from 22 bacterial species are provided in FIG. 7 of United States Patent Publication No. 2003/0044429, which is hereby incorporated by reference in its entirety. Therefore, the sequence differences between species is included within the meaning of the term. In certain embodiments a flagellin-based agent in accordance with the present disclosure includes an amino acid sequence having at least 80% identity, or at least 85% identity, or at least 90% identity, or at least 95% identity, or at least 97% identity, or at least 98% identity, or at least 99% identity, or 100% identity with one or more of the flagellin from 22 bacterial species provided in FIG. 7 of United States Patent Publication No. 2003/0044429. The amino acid sequences of the conserved amino and carboxy terminus (important for TLR5 activity) from 21 species of bacteria are provided in FIG. 24A and 24B of U.S. Pat. No. 8,007,812, which is hereby incorporated by reference in its entirety.

In certain embodiments, a flagellin-based agent in accordance with the present disclosure includes a fragment of a flagellin protein or a flagellin-based agent. In some embodiments a flagellin based-agent or fragment thereof has activity as a TLR5 agonist. In various embodiments,

In some embodiments, the TLR5 agonist is a Salmonella flagellin protein, e.g. a recombinant Salmonella flagellin protein. In some embodiments, the TLR5 agonist is a Salmonella dublin flagellin protein, e.g. a recombinant Salmonella dublin flagellin protein. In various embodiments, the Salmonella dublin flagellin protein has the amino acid sequence of SEQ ID NO: 27, as shown below:

(SEQ ID NO: 27)
MAQVINTNSLSLLTQNNLNKSQSSLSSAIERLSSGLRINSAKDDAAGQA
IANRFTSNIKGLTQASRNANDGISIAQTTEGALNEINNNLQRVRELSVQ
ATNGINSDSDLKSIQDEIQQRLEEIDRVSNQTQFNGVKVLSQDNQMKIQ
VGANDGETITIDLQKIDVKSLGLDGFNVNGPKEATVGDLKSSFKNVTGY
DTYAAGADKYRVDINSGAVVTDAAAPDKVYVNAANGQLTTDDAENNTAV
DLFKTTKSTAGTAEAKAIAGAIKGGKEGDTFDYKGVTFTIDTKTGDDGN
GKVSTTINGEKVTLTVADIATGAADVNAATLQSSKNVYTSVVNGQFTFD
DKTKNESAKLSDLEANNAVKGESKITVNGAEYTANATGDKITLAGKTMF
IDKTASGVSTLINEDAAAAKKSTANPLASIDSALSKVDAVRSSLGAIQN
RFDSAITNLGNTVTNLNSARSRIEDADYATEVSNMSKAQILQQAGTSVL
AQANQVPQNVLSLLR.

In some embodiments, the present invention contemplates use of a TLR5 agonist comprising a polypeptide having an amino acid sequence having at least about 80%, at least about 85%, at least about 87%, at least about 90%, at least about 93% at least about 95%, or at least about 96%, or at least about 97% or at least about 98%, or at least about 99%, or 100% sequence identity to SEQ ID NO: 27. In various embodiments, the polypeptide having an amino acid sequence does not comprise a His tag.

In some embodiments of the methods provided herein, the TLR5 agonist that is not fused to a pathogenic protein antigen is entolimod (CBLB502). Entolimod (CBLB502) is a flagellin-related polypeptide (see, e.g., FIG. 7 of U.S. Patent Publication No. 2003/0044429, the contents of which are incorporated herein by reference in their entirety). As used herein“entolimod” (aka “CBLB502”) refers to a polypeptide which has the sequence of SEQ ID NO: 1 of WIPO Patent Application WO/2016/109002 (hereby incorporated by reference in its entirety), as shown below:

(SEQ ID NO: 1)
MRGSHHHHHHGMASMTGGQQMGRDLYDDDDKDPMAQVINTNSLSLLTQN
NLNKSQSSLSSAIERLSSGLRINSAKDDAAGQAIANRFTSNIKGLTQAS
RNANDGISIAQTTEGALNEINNNLQRVRELSVQATNGTNSDSDLKSIQD
EIQQRLEEIDRVSNQTQFNGVKVLSQDNQMKIQVGANDGETITIDLQKI
DVKSLGLDGFNVNSPGISGGGGGILDSMGTLINEDAAAAKKSTANPLAS
IDSALSKVDAVRSSLGAIQNRFDSAITNLGNTVTNLNSARSRIEDADYA
TEVSNMSKAQILQQAGTSVLAQANQVPQNVLSLLR.

In some embodiments, the present invention contemplates use of a TLR5 agonist comprising a polypeptide having an amino acid sequence having at least about 80%, at least about 85%, at least about 87%, at least about 90%, at least about 93% at least about 95%, or at least about 96%, or at least about 97% or at least about 98%, or at least about 99%, or 100% sequence identity to SEQ ID NO: 1. In various embodiments, the polypeptide having an amino acid sequence does not comprise a His tag.

In some embodiments of the aspects and embodiments provided herein, the TLR5 agonist that is not fused to a pathogenic protein is a flagellin-based agent comprising a polypeptide having an amino acid sequence having at least 80% identity, or at least 85% identity, or at least 90% identity, or at least 95% identity, or at least 97% identity, or at least 98% identity, or at least 99% identity, or 100% identity with one or more of CBLB502-S33ML (SEQ ID NO: 35 of WO/2016/019034), CBLB502-485CT (CBLB533, SEQ ID NO: 71 of WO/2016/019034), CBLB502-S33MX (CBLB543, SEQ ID NO: 150 of WO/2016/019034), CBLB502-S33 (SEQ ID NO: 17 of WO/2016/019034), Mutant 33ML (SEQ ID NO: 42 of WO 2016/019034) of International Patent Application WO 2016/019034 (hereby incorporated by reference in its entirety), as shown below, respectively:

CBLB502-S33ML (SEQ ID NO: 35 of WO/2016/019034)
(SEQ ID NO: 2)
MSGLRINSAKDDAAGQAIANRFTSNIKGLTQASRNANDGISIAQTTEGA
LNEINNNLQRVRELSVQATNGTNSDSDLKSIQDEIQQRLEEIDRVSNQT
QFNGVKVLSQDNQMKIQVGANDGETITIDLQKIDVKSLGLDGFNVNSPG
ISGGGGGILDSMGTLINEDAAAAKKSTANPLASIDSALSKVDAVRSSLG
AIQNRFDSAITNLGNTVTNLNSARSRIEDADYATEVSNMSKAQILQQAG
TSVLAQANQVPQNVLSLLVPRGSHHHHHHG;
CBLB502-485CT (CBLB533, SEQ ID NO: 71 of WO/2016/
019034)
(SEQ ID NO: 3)
MSGLRINSAKDDAAGQAIANRFTSNIKGLTQASRNANDGISIAQTTEGA
LNEINNNLQRVRELSVQATNGTNSDSDLKSIQDEIQQRLEEIDRVSNQT
QFNGVKVLSQDNQMKIQVGANDGETITIDLQKIDVKSLGLDGFNVNSPG
STANPLASIDSALSKVDAVRSSLGAIQNRFDSAITNLGNTVTNLNSARS
RIEDADYATEVSNMSKAQILQQAGLVPRGSHHHHHHG;
CBLB502-S33MX (CBLB543, SEQ ID NO: 150 of WO/2016/
019034)
(SEQ ID NO: 4)
MSGLRINSAKDDAAGQAIANRFTSNIKGLTQASRNAADGISIAQTTEGA
LNEINNNLQRVRELSVQATAGANADAALKAIQAEIQQRLEEIDRVSQQT
QAAAVKVLSQDNAMAIQVGANDGAAITIDLQKIDVKSLGLDGFNVNSPG
STANPLASIDSALSKVDAVRSSLGAIQNRFDSAITNLGNTVTNLNSARS
RIEDADYATEVSQMSKAQILQQAGTSVLAQANQVPQNVLSLLVPRGSHH
HHHHG;
CBLB502-S33 (SEQ ID NO: 17 of WO/2016/019034)
(SEQ ID NO: 5)
MRGSHHHHHHGMASMTGGQQMGRDLYDLVPRGSAKDPSGLRINSAKDDA
AGQAIANRFTSNIKGLTQASRNANDGISIAQTTEGALNEINNNLQRVRE
LSVQATNGTNSDSDLKSIQDEIQQRLEEIDRVSNQTQFNGVKVLSQDNQ
MKIQVGANDGETITIDLQKIDVKSLGLDGFNVNSPGISGGGGGILDSMG
TLINEDAAAAKKSTANPLASIDSALSKVDAVRSSLGAIQNRFDSAITNL
GNTVTNLNSARSRIEDADYATEVSNMSKAQILQQAGTSVLAQANQVPQN
VLSLLR;
and
Mutant 33ML (SEQ ID NO: 42 of WO/2016/019034)
(SEQ ID NO: 6)
MSGLRINSAKDDAAGQAIANRFTSNIKGLTQASRNANDGISIAQTTEGA
LNEINNNLQRVRELSVQATNGTNSDSDLKSIQDEIQQRLEEIDRVSNQT
QFNGVKVLSQDNQMKIQVGANDGETITIDLQKIDVKSLGLDGFNVNSPG
STANPLASIDSALSKVDAVRSSLGAIQNRFDSAITNLGNTVTNLNSARS
RIEDADYATEVSNMSKAQILQQAGTSVLAQANQVPQNVLSLLVPRGSHH
HHHHG.

In some embodiments, the present invention contemplates use of a TLR5 agonist comprising a polypeptide having an amino acid sequence having at least about 80%, at least about 85%, at least about 87%, at least about 90%, at least about 93% at least about 95%, or at least about 96%, or at least about 97% or at least about 98%, or at least about 99%, or 100% sequence identity to one or more of SEQ ID NOs: 2-6. In various embodiments, the polypeptide having an amino acid sequence does not comprise a His tag.

In some embodiments of the aspects and embodiments provided herein, the TLR5 agonist that is not fused to a pathogenic protein is a flagellin-based agent comprising a polypeptide having an amino acid sequence having at least 80% identity, or at least 85% identity, or at least 90% identity, or at least 95% identity, or at least 97% identity, or at least 98% identity, or at least 99% identity or 100% identity with one or more of SEQ ID NOs: 243-252 of International Patent Application WO 2016/019134 (hereby incorporated by reference in its entirety), as shown below, respectively:

SEQ ID NO: 243 of WO 2016/019134
(SEQ ID NO: 7)
MGHHHHHHSGMEEFNMRINTNVAAMNTYSRLTAANTAKSNSLAKLSSGL
RINKAGDDAAGLAISEKMKSQIGGLTQAKRNAQDGISLVQTAEGALNET
HSILERMRDLAVQGSNGTLISSDRGSINKELKALHQELTRISNITEFNT
QKLFSQTKQKSVIFTFQIGANAGQTLSVAITAMSGEALLVSTDAKFSLN
AAGTNAGAMIKSIDAAIAKVSDQRADLGAVQNRLEHTINNLTATNENLS
DANSRIRDVDMAEEMMTFTKSNILSQAATSMLAQANAMPNSVLNLLQG;
SEQ ID NO: 244 of WO 2016/019134
(SEQ ID NO: 8)
MGHHHHHHSGMRINHNISALNAWRNIDQTQYSMSKTLERLSSGLRINRA
GDDAAGLAISEKMRGQIKGLNMAIKNAQDAISLIQTAEGALTEVHSILQ
RMRELAVQAASDTNTNVDREQIQKEIDQLREEIDRIARTTEFNIKKLLD
GKLEGFRSQVDAKVVTGGNINVQLGTVSSKAVEGTYVIEVGAAERAIMV
VDAAIHRVSTARAALGAIQNRLEHTISNLGVAAENLTAAESRIRDADMA
KEMMEFTKQQILLQSSMAMLAQSNTLPQNVLQLMR;
SEQ ID NO: 245 of WO 2016/019134
(SEQ ID NO: 9)
MGHHHHHHSGLNMAIKNAQDAISLIQTAEGALTEVHSILQRMRELAVQA
ASDTNTNVDREQIQKEIDQLREEIDRIARTTEFNIKKLLDGKLEGFRSQ
VDAKVVTGGNINVQLGTVSSKAVEGTYVIEVGAAERAIMVVDAAIHRVS
TARAALGAIQNRLEHTISNLG;
SEQ ID NO: 246 of WO 2016/019134
(SEQ ID NO: 10)
MGHHHHHHSGMSLRINNNIEALNAWRALNSTSNALQKSMEKLSSGLRIN
RAGDDAAGLAISEKLRAQIRGLNQAIRNAQDGISLIQTAEGGLSEIQNI
LQRMRELGVQAANGTLNNQDISAITTELNQLFNEIDRIAGATEFNIKNL
LAVSTGLVVTLQVGANAGQVIAFTIDNAGTASLGLSSADLAINDNASAS
AFISKVDSALQKVSTYRANLGSIQNRLEHTIANLGIASENLSASESRIR
DVDMAAEMMNFTKNQILQQAGVAILAQANQAPQAVLQLLR;
SEQ ID NO: 247 of WO 2016/019134
(SEQ ID NO: 11)
MGHHHHHHSGLNQAIRNAQDGISLIQTAEGGLSEIQNILQRMRELGVQA
ANGTLNNQDISAITTELNQLFNEIDRIAGATEFNIKNLLAVSTGLVVTL
QVGANAGQVIAFTIDNAGTASLGLSSADLAINDNASASAFISKVDSALQ
KVSTYRANLGSIQNRLEHTIANLG;
SEQ ID NO: 248 of WO 2016/019134
(SEQ ID NO: 12)
MGHHHHHHSGLNQAIRNAQDGISLIQTAEGGLSEIQNILQRMRELGVQA
ANGTLNNQDISAITTELNQLFNEIDRIAGATEFNTKNLLAAGTASLGLS
SADLAINDNASASAFISKVDSALQKVSTYRANLGSIQNRLEHTIANLG;
SEQ ID NO: 249 of WO 2016/019134
(SEQ ID NO: 13)
MGHHHHHHSASAFISKVDSALQKVSTYRANLGSIQNRLEHTIANLGPDG
LNQAIRNAQDGISLIQTAEGGLSEIQNILQRMRELGVQAANGTLNNQDI
SAITTELNQLFNEIDRIA;
SEQ ID NO: 250 of WO 2016/019134
(SEQ ID NO: 14)
MGHHHHHHSNNQDISAITTELNQLFNEIDRIAGATGSGGLSEIQNILQR
MRELGVQAANGTLNGGSASAFISKVDSALQKVSTYRANLGSIQNRLEHT
IANLG;
SEQ ID NO: 251 of WO 2016/019134
(SEQ ID NO: 15)
MGHHHHHHSGLAQASRNAQDAISIAQTAEGALDETQSILQRVRELGVQG
ANGTLTADDINALQAEVDQLIAEIDRIAGATEFNTQNLLDGSFTTKAFQ
VGANSGQNMTLTIGKMDTTTLGLSSADLAINDNAFANGAISTVDSALQK
VSAERAKLGAIQNRLEHTIANLG;
and
SEQ ID NO: 252 of WO 2016/019134
(SEQ ID NO: 16)
MGHHHHHHSGLAQASRQAQDAISIAQTAEGALDETQSILQRVRELGVQG
ADGTLTADDIDALQAEVDQLIAEIDRIAGATEFATQKLLDGSFTTKAFQ
VGAASGQDVTLTIGKVDTTTLGLSSADLAIDSAAFADGAISTVDSALQK
VSAERAKLGAIQNRLEHTIAQLG.

In some embodiments, the present invention contemplates use of a TLR5 agonist comprising a polypeptide having an amino acid sequence having at least about 80%, at least about 85%, at least about 87%, at least about 90%, at least about 93% at least about 95%, or at least about 96%, or at least about 97% or at least about 98%, or at least about 99%, or 100% sequence identity to one or more of SEQ ID NOs: 7-16. In various embodiments, the polypeptide having an amino acid sequence does not comprise a His tag.

In some embodiments of the aspects and embodiments provided herein, the TLR5 agonist that is not fused to a pathogenic protein is a flagellin-based agent comprising a polypeptide having an amino acid sequence having at least 80% identity, or at least 85% identity, or at least 90% identity, or at least 95% identity, or at least 97% identity, or at least 98% identity, or at least 99% identity or 100% identity with one or more of SEQ ID NOs: 10, 12, 30, 32, 34, 36, 38, 40, 42, or 44 of International Patent Application WO 2006/069198 (hereby incorporated by reference in its entirety), as shown below, respectively:

SEQ ID NO: 10 of WO 2006/069198
(SEQ ID NO: 17)
MRGSHHHHHHGMASMTGGQQMGRDLYDDDDKDPMAQVINTNSLSLLTQN
NLNKSQSSLSSAIERLSSGLRINSAKDDAAGQAIANRFTSNIKGLTQAS
RNANDGISIAQTTEGALNEINNNLQRVRELSVQATNGTNSDSDLKSIQD
EIQQRLEEIDRVSNQTQFNGVKVLSQDNQMKIQVGANDGETITIDLQKI
DVKSLGLDGFNVNSPGISGGGGGILDSMGTLINEDAAAAKKSTANPLAS
IDSALSKVDAVRSSLGAIQNRFDSAITNL;
SEQ ID NO: 12 of WO 2006/069198
(SEQ ID NO: 17)
MRGSHHHHHHGMASMTGGQQMGRDLYDDDDKDPFTSNIKGLTQASRNAN
DGISIAQTTEGALNEINNNLQRVRELSVQATNGTNSDSDLKSIQDEIQQ
RLEEIDRVSNQTQFNGVKVLSQDNQMKIQVGANDGETITIDLQKIDVKS
LGLDGFNVNSPGISGGGGGILDSMGTLINEDAAAAKKSTANPLASIDSA
LSKVDAVRSSLGAIQNRFDSAITNLGNTVTNLNSARSRIEDADYATEVS
NMSKAQILQQAGTSVLAQANQVPQNVLSLLR;
SEQ ID NO: 30 of WO 2006/069198
(SEQ ID NO: 17)
MRGSHHHHHHGMASMTGGQQMGRDLYDDDDKDPMAQVINTNSLSLLTQN
NLNKSQSSLSSAIERLSSGLRINSAKDDAAGQAIANRFTSNIKGLTQAS
RNANDGISIAQTTEGALNEINNNLQRVRELSVQATNGTNSDSDLKSIQD
EIQQRLEEIDRVSNQTQFNGVKVLSQDNQMKIQVGANDGETITIDLQKI
DVKSLGLIPGISGGGGGILDSMGTLINEDAAAAKKSTANPLASIDSALS
KVDAVRSSLGAIQNRFDSAITNLGNTVTNLNSARSRIEDADYATEVSNM
SKAQILQQAGTSVLAQANQVPQNVLSLLR;
SEQ ID NO: 32 of WO 2006/069198
(SEQ ID NO: 17)
MRGSHHHHHHGMASMTGGQQMGRDLYDDDDKDPFTSNIKGLTQASRNAN
DGISIAQTTEGALNEINNNLQRVRELSVQATNGTNSDSDLKSIQDEIQQ
RLEEIDRVSNQTQFNGVKVLSQDNQMKIQVGANDGETITIDLQKIDVKS
LGLIPGISGGGGGILDSMGTLINEDAAAAKKSTANPLASIDSALSKVDA
VRSSLGAIQNRFDSAITNLGNTVTNLNSARSRIEDADYATEVSNMSKAQ
ILQQAGTSVLAQANQVPQNVLSLLR;
SEQ ID NO: 34 of WO 2006/069198
(SEQ ID NO: 17)
MRGSHHHHHHGMASMTGGQQMGRDLYDDDDKDPMAQVINTNSLSLLTQN
NLNKSQSSLSSAIERLSSGLRINSAKDDAAGQAIANRFTSNIKGLTQAS
RNANDGISIAQTTEGALNEINNNLQRVRELSVQATNGTNSDSDLKSIQD
EIQQRLEEIDRVSNQTQFNGVKVLSQDNQMKIQVGANDGETITIDLQKI
IPGISGGGGGILDSMGTLINEDAAAAKKSTANPLASIDSALSKVDAVRS
SLGAIQNRFDSAITNLGNTVTNLNSARSRIEDADYATEVSNMSKAQILQ
QAGTSVLAQANQVPQNVLSLLR;
SEQ ID NO: 36 of WO 2006/069198
(SEQ ID NO: 17)
MRGSHHHHHHGMASMTGGQQMGRDLYDDDDKDPFTSNIKGLTQASRNAN
DGISIAQTTEGALNEINNNLQRVRELSVQATNGTNSDSDLKSIQDEIQQ
RLEEIDRVSNQTQFNGVKVLSQDNQMKIQVGANDGETITIDLQKIIPGI
SGGGGGILDSMGTLINEDAAAAKKSTANPLASIDSALSKVDAVRSSLGA
IQNRFDSAITNLGNTVTNLNSARSRIEDADYATEVSNMSKAQILQQAGT
SVLAQANQVPQNVLSLLR;
SEQ ID NO: 38 of WO 2006/069198
(SEQ ID NO: 17)
MRGSHHHHHHGMASMTGGQQMGRDLYDDDDKDPMAQVINTNSLSLLTQN
NLNKSQSSLSSAIERLSSGLRINSAKDDAAGQAIANRFTSNIKGLTQAS
RNANDGISIAQTTEGALNEINNNLQRVRELSVQATNGTNSDSDLKSIQD
EIQQRLEEIDRVSNQTQFNGVKVLSQDNQMKIQVGANDGETITIDLQKI
DVKSLGLIPGISGGGGGILDSMGTLINEDAAAAKKSTANPLASIDSALS
KVDAVRSSLGAIQNRFDSAITNL;
SEQ ID NO: 40 of WO 2006/069198
(SEQ ID NO: 17)
MRGSHHHHHHGMASMTGGQQMGRDLYDDDDKDPMAQVINTNSLSLLTQN
NLNKSQSSLSSAIERLSSGLRINSAKDDAAGQAIANRFTSNIKGLTQAS
RNANDGISIAQTTEGALNEINNNLQRVRELSVQATNGTNSDSDLKSIQD
EIQQRLEEIDRVSNQTQFNGVKVLSQDNQMKIQVGANDGETITIDLQKI
IPGISGGGGGILDSMGTLINEDAAAAKKSTANPLASIDSALSKVDAVRS
SLGAIQNRFDSAITNL;
SEQ ID NO: 42 of WO 2006/069198
(SEQ ID NO: 17)
MRGSHHHHHHGMASMTGGQQMGRDLYDDDDKDPMAQVINTNSLSLLTQN
NLNKSQSSLSSAIERLSSGLRINSAKDDAAGQAIANRFTSNIKGLTQAS
RNANDGISIAQTTEGALNEINNNLQRVRELSVQATNGTNSDSDLKSIQD
EIQQRLEEIDRVSNQIPGISGGGGGILDSMGTLINEDAAAAKKSTANPL
ASIDSALSKVDAVRSSLGAIQNRFDSAITNLGNTVTNLNSARSRIEDAD
YATEVSNMSKAQILQQAGTSVLAQANQVPQNVLSLLR;
and
SEQ ID NO: 44 of WO 2006/069198
(SEQ ID NO: 17)
MRGSHHHHHHGMASMTGGQQMGRDLYDDDDKDPFTSNIKGLTQASRNAN
DGISIAQTTEGALNEINNNLQRVRELSVQATNGTNSDSDLKSIQDEIQQ
RLEEIDRVSNQIPGISGGGGGILDSMGTLINEDAAAAKKSTANPLASID
SALSKVDAVRSSLGAIQNRFDSAITNLGNTVTNLNSARSRIEDADYATE
VSNMSKAQILQQAGTSVLAQANQVPQNVLSLLR.

In some embodiments, the present invention contemplates use of a TLR5 agonist comprising a polypeptide having an amino acid sequence having at least about 80%, at least about 85%, at least about 87%, at least about 90%, at least about 93% at least about 95%, or at least about 96%, or at least about 97% or at least about 98%, or at least about 99%, or 100% sequence identity to one or more of SEQ ID NOs: 17-26. In various embodiments, the polypeptide having an amino acid sequence does not comprise a His tag.

Examples of the pathogenic protein antigen that in some embodiments would not be fused to a TLR5 agonist and/or flagellin based agent as described herein include an α-helix domain of surface protein A (PspA) and pneumococcal surface protein A (PsaA) of Streptococcus pneumonia; subunit hemagglutinin (HA) and neuraminidase (NA) of influenza virus; and spike (S) protein of severe acute respiratory syndrome virus (SARS virus), and the like.

### Dosage, Administration and Pharmaceutical Formulation

In embodiments, a pharmaceutical preparation of TLR5 agonist is used in the variousmethods and, in some embodiments, it may be in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents. Some pharmaceutical preparations can deliver the compounds of the disclosure in a sustained release formulation.

A of TLR5 agonist according to the invention, the dosage form may optionally be a liquid dosage form. Solutions can be prepared in water suitably mixed with a surfactant such as hydroxypropylcellulose or an emulsifier such as polysorbate. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. Formulations optionally contain excipients including, but not limited to, a buffering agents, an anti-oxidant, a stabilizer, a carrier, a diluent, and an agent for pH adjustment. The pharmaceutical forms suitable for injectable use include sterile aqueous solutions or dispersion and sterile powders for the extemporaneous preparation of sterile injectable solutions or dispersions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl, or benzyl alcohol; alkyl parabens such as methyl or propyl paraben; catechol; resorcinol; cyclohexanol; 3-pentanol; and m-cresol); low molecular weight (less than about 10 residues) polypeptides; proteins such as serum, albumin, gelatin, or immunoglobulins; hydrophilic polymers such as polyvinylpyrrolidone; amino acids such as glycine, glutamine, asparagine, histidine, arginine or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN, PLURONICS or polyethylene glycol (PEG).

In treatment, the dose of of TLR5 agonist optionally ranges from about 0.0001 mg/kg to about 100 mg/kg, about 0.01 mg/kg to about 5 mg/kg, about 0.15 mg/kg to about 3 mg/kg, 0.5 mg/kg to about 2 mg/kg and about 1 mg/kg to about 2 mg/kg of the subject's body weight. In other embodiments the dose ranges from about 100 mg/kg to about 5 g/kg, about 500 mg/kg to about 2 mg/kg and about 750 mg/kg to about 1.5 g/kg of the subject's body weight. For example, depending on the type and severity of the disease or disorder, about 1 .mu.g/kg to 15 mg/kg (e.g., 0.1-20 mg/kg) of agent is a candidate dosage for administration to the patient, whether, for example, by one or more separate administrations, or by continuous infusion. A typical daily dosage is in the range from about 1 mg/kg to 100 mg/kg or more, depending on the factors mentioned above. For repeated administrations over several days or longer, depending on the condition, the treatment is sustained until a desired suppression of disease or disorder symptoms occurs. However, other dosage regimens may be useful. Unit doses can be in the range, for instance of about 5 mg to 500 mg, such as 50 mg, 100 mg, 150 mg, 200 mg, 250 mg and 300 mg. The progress of therapy is monitored by conventional techniques and assays.

In some embodiments, a TLR5 agonist, e.g. flagellin or flagellin-based agent (such as entolimod) is administered to a human patient at an effective amount (or dose) of less than about 1 μg/kg, for instance, about 0.35 to about 0.75 μg/kg or about 0.40 to about 0.60 μg/kg. In some embodiments, the dose of a flagellin or flagellin-based agent (such as entolimod) is about 0.35 μg/kg, or about 0.40 μg/kg, or about 0.45 μg/kg, or about 0.50 μg/kg, or about 0.55 μg/kg, or about 0.60 μg/kg, or about 0.65 μg/kg, or about 0.70 μg/kg, or about 0.75 μg/kg, or about 0.80 μg/kg, or about 0.85 μg/kg, or about 0.90 μg/kg, or about 0.95 μg/kg or about 1 μg/kg. In various embodiments, the absolute dose of a flagellin or flagellin-based agent (such as entolimod) is about 2 μg/subject to about 45 μg/subject, or about 5 to about 40, or about 10 to about 30, or about 15 to about 25 μg/subject. In some embodiments, the absolute dose of a flagellin or flagellin-based agent (such as entolimod) is about 20 μg, or about 30 μg, or about 40 μg.

In various embodiments, the dose of TLR5 agonist, e.g. a flagellin or flagellin-based agent (such as entolimod) may be determined by the human patient's body weight. For example, an absolute dose of a flagellin or flagellin-based agent (such as entolimod) of about 2 μg for a pediatric human patient of about 0 to about 5 kg (e.g. about 0, or about 1, or about 2, or about 3, or about 4, or about 5 kg); or about 3 μg for a pediatric human patient of about 6 to about 8 kg (e.g. about 6, or about 7, or about 8 kg), or about 5 μg for a pediatric human patient of about 9 to about 13 kg (e.g. 9, or about 10, or about 11, or about 12, or about 13 kg); or about 8 μg for a pediatric human patient of about 14 to about 20 kg (e.g. about 14, or about 16, or about 18, or about 20 kg), or about 12 μg for a pediatric human patient of about 21 to about 30 kg (e.g. about 21, or about 23, or about 25, or about 27, or about 30 kg), or about 13 μg for a pediatric human patient of about 31 to about 33 kg (e.g. about 31, or about 32, or about 33 kg), or about 20 μg for an adult human patient of about 34 to about 50 kg (e.g. about 34, or about 36, or about 38, or about 40, or about 42, or about 44, or about 46, or about 48, or about 50 kg), or about 30 μg for an adult human patient of about 51 to about 75 kg (e.g. about 51, or about 55, or about 60, or about 65, or about 70, or about 75 kg), or about 45 μg for an adult human patient of greater than about 114 kg (e.g. about 114, or about 120, or about 130, or about 140, or about 150 kg).

In certain embodiments, a TLR5 agonist, e.g. a flagellin or flagellin-based agent (such as entolimod) in accordance with the methods provided herein is administered subcutaneously (s.c.), intraveneously (i.v.), intramuscularly (i.m.), intranasally or topically. Administration of a flagellin or flagellin-based agent (such as entolimod) described herein can, independently, be one to four times daily or one to four times per month or one to six times per year or once every two, three, four or five years. Administration can be for the duration of one day or one month, two months, three months, six months, one year, two years, three years, and may even be for the life of the human patient. The dosage may be administered as a single dose or divided into multiple doses. In some embodiments, a flagellin or flagellin-based agent (such as entolimod) is administered about 1 to about 3 times (e.g. 1, or 2 or 3 times). In some embodiments, a flagellin or flagellin-based agent (such as entolimod) is administered once.

In some embodiments of the methods provided herein, TLR5 agonist, e.g. a flagellin or flagellin-based agent (such as entolimod) is administered in one or more cycles. In certain embodiments of the methods as provided herein, a TLR5 agonist, e.g. a flagellin or flagellin-based agent (such as entolimod) is administered in one or more cycles in which a cycle involves dosing a patient once per day for one day; or once a day for two days; or once a day for three days; or once a day for four days; or once a day for five days. In certain embodiments of the methods as provided herein, a TLR5 agonist, e.g. a flagellin or flagellin-based agent (such as entolimod) is administered in one or more cycles as provided herein, and wherein no more than 5 cycles are administered per year; or no more than 3 cycles are administered per year; or no more than 2 cycles are administered per year.

Various modes of administration of a TLR5 agonist, e.g. a flagellin or flagellin-based agent (such as entolimod) are contemplated herein. In one embodiment, a TLR5 agonist, e.g. a flagellin or flagellin-based agent (such as entolimod) is administered parenterally. In some embodiments, a TLR5 agonist, e.g. a flagellin or flagellin-based agent (such as entolimod) is administered by injection, e.g. intramuscular injection. In some embodiments, a TLR5 agonist, e.g. a flagellin or flagellin-based agent (such as entolimod) is by a single intramuscular injection. In some embodiments, administration is accomplished using a kit as described herein (e.g. via a unit dose form, e.g. a pre-loaded (a.k.a. pre-dosed or pre-filled) syringe or a pen needle injector (injection pen)).

### Kits

The invention provides kits that can simplify the administration of any agent described herein. An illustrative kit of the invention comprises any composition described herein in unit dosage form. In one embodiment, the unit dosage form is a container, such as a pre-filled syringe, which can be sterile, containing any agent described herein and a pharmaceutically acceptable carrier, diluent, excipient, or vehicle. The kit can further comprise a label or printed instructions instructing the use of any agent described herein. The kit may also include a lid speculum, topical anesthetic, and a cleaning agent for the administration location. The kit can also further comprise one or more additional agent described herein. In one embodiment, the kit comprises a container containing an effective amount of a composition of the invention and an effective amount of another composition, such those described herein.

### Definitions

With respect to the agents described herein, the terms “modulate” and “modulation” refers to the upregulation (i.e., activation or stimulation) or downregulation (i.e., inhibition or suppression) of a response. A “modulator” is an agent, compound, or molecule that modulates, and may be, for example, an agonist, antagonist, activator, stimulator, suppressor, or inhibitor. The terms “inhibit”, “reduce”, remove as used herein refer to any inhibition, reduction, decrease, suppression, downregulation, or prevention in expression, activity or symptom and include partial or complete inhibition of activity or symptom. Partial inhibition can imply a level of expression, activity or symptom that is, for example, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% of the uninhibited expression, activity or symptom. The terms “eliminate” or “eradicate” indicate a complete reduction of activity or symptom.

As used herein, the term “a disorder” or “a disease” refers to any derangement or abnormality of function; a morbid physical or mental state. See Dorland's Illustrated Medical Dictionary, (W.B. Saunders Co. 27th ed. 1988).

As used herein, the term “treating” or “treatment” of any disease or disorder refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

As used herein, the term “abnormal” refers to an activity or feature which differs from a normal activity or feature. As used herein, the term “abnormal activity” refers to an activity which differs from the activity of the wild-type or native gene or protein, or which differs from the activity of the gene or protein in a healthy subject. The abnormal activity can be stronger or weaker than the normal activity. In one embodiment, the “abnormal activity” includes the abnormal (either over- or under-) production of mRNA transcribed from a gene. In another embodiment, the “abnormal activity” includes the abnormal (either over- or under-) production of polypeptide from a gene. In another embodiment, the abnormal activity refers to a level of a mRNA or polypeptide that is different from a normal level of the mRNA or polypeptide by about 15%, about 25%, about 35%, about 50%, about 65%, about 85%, about 100% or greater. In some embodiments, the abnormal level of the mRNA or polypeptide can be either higher or lower than the normal level of the mRNA or polypeptide. Yet in another embodiment, the abnormal activity refers to functional activity of a protein that is different from a normal activity of the wild-type protein. In some embodiments, the abnormal activity can be stronger or weaker than the normal activity. In some embodiments, the abnormal activity is due to the mutations in the corresponding gene, and the mutations can be in the coding region of the gene or non-coding regions such as transcriptional promoter regions. The mutations can be substitutions, deletions, insertions.

“Therapeutically effective amount” as used herein means the amount of a compound or composition (such as described herein) that causes at least one desirable change in a cell, population of cells, tissue, individual, patient or the like. In some embodiments a therapeutically effective amount as used herein means the amount of a compound or composition (such as described herein) that prevents or provides a clinically significant change in a disease or disorder or condition (e.g., reduce by at least about 30 percent, at least about 50 percent, or at least about 90 percent) or in one or more features of a disease or disorder or condition described herein.

### EXAMPLES

The present disclosure will be further described in the following examples, which do not limit the scope of any invention or inventions described in the claims.

### Example 1: Pharmacological Stimulation of TLR5 Improves Quality of Life and Reduces Frailty

This example describes a pharmacological flagellin-based agent and method of its use to prevent aging-related frailty and extend healthy life (“healthspan”) and longevity (“lifespan”).

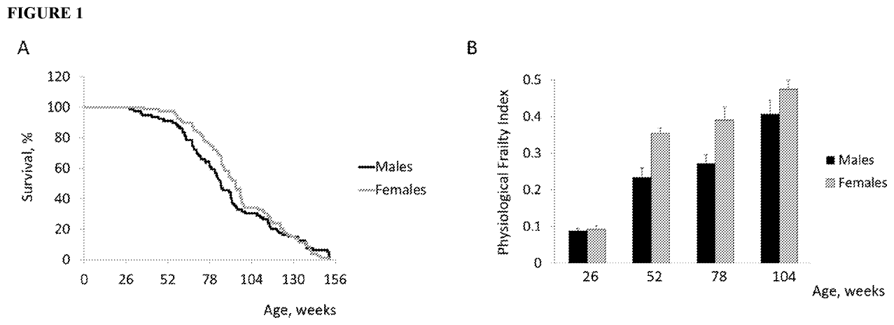

Preclinical model of aging. General decline of the majority of physiological functions, regenerating capabilities, impaired wound healing, gradual elevation of the risk of a variety of diseases or disorders, increase in systemic sterile inflammation occur during lifetime of all mammals leads to acquisition of a combination of symptoms cumulatively defined as frailty. Increase in frailty observed in healthy animals and humans that is not provoked or accelerated by any particular pathology is named chronological aging. The severity of this condition grows with time and reaches critical degrees of severity when the organism approaches age that is close to its natural genetically determined lifespan. Chronological aging of laboratory mouse is a well-accepted model of human aging commonly used in the field of gerontology. Natural lifespan of most of strains of mice is about 2 years. Each strain is characterized by its prevalent spectrum of age-related diseases or disorders, which becomes a major cause of death, a phenomenon that may jeopardize other manifestations of aging and complicate studies of frailty. To minimize the influence of this factor, we used for our studies outbred NIH Swiss mice that are naturally less prone to genetic predisposition to certain specific pathologies. We have characterized the longevity of both genders of NIH Swiss mice that are maintained under conditions of minimized risk of the health risks (infections, poisoning, trauma, stress, etc.) other than age-related frailty. These conditions are provided in the Department of Laboratory Animals of Roswell Park Cancer Institute, where animals are maintained according to the Animal Welfare Guidelines of NIH. Under these conditions, animals have the highest chance to fully realize their natural lifespan and die from frailty that reflects endogenous processes of natural chronological aging (FIG. 1). The adequacy of this model was confirmed by its capability to reveal the biological effect of factors that are known to modulate longevity as shown in FIG. 7: treatment of mice with mTOR inhibitor rapamycin was shown to extend the lifespan of mice. Indeed, the results of treatment with mTOR inhibitor rapamycin appear to have the polar opposite effect of the results shown regarding administration of flagellin or entolimod.

Objective quantitative assessment of biological age was done by determination of a so-called “frailty index” (FI), a parameter which reflects the scale of accumulation of age-related deficits. Commonly used in gerontological clinics, FI was adapted to laboratory animals and was calculated for each animal as a function of the degrees of deviation of multiple measurable physiological and biochemical parameters from those of young and healthy animals. The resulting number, which we term “Physiological Frailty Index (PFI), gradually grows with life and reflects the biological age of animal. PFI is expressed as a score from “0” (no deficits, within the range of the reference group) to “1” (extreme deficits).

Flagellin of Salmonella. Flagellin, a bacterial protein, the major component of bacterial flagella, is the only known agonist of innate immunity receptor TLR5. Salmonella flagellin was synthesized as a recombinant protein in E. coli and affinity purified on Ni-containing column for its His tag followed by other purification steps (e.g., polymyxin column—to get rid of endotoxin) as previously described (Burdelya, L. G. et al. An Agonist of Toll-Like Receptor 5 Has Radioprotective Activity in Mouse and Primate Models. Science 320, 226-230 (2008)). The quality of the resulting product was controlled using a series of functional assays involving a panel of reporter cell lines expressing individual TLRs: it was capable of activating NF-kappaB signaling only in the cells expressing TLR5 but not other TLRs. Flagellin is stored in solution as deeply frozen aliquots.

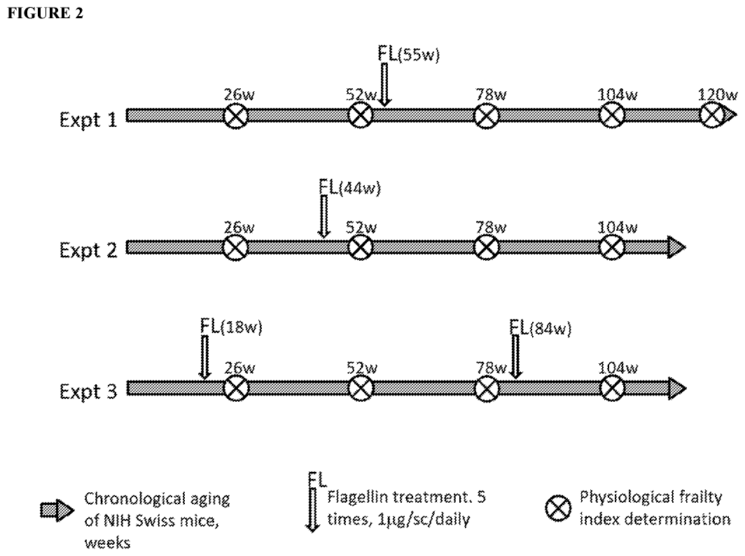

Experimental design and rationale. Animals were maintained under strictly controlled conditions of temperature, day-night switch, healthy balanced diet, constant access to drinking water, sterile food and air. Their PFI was measured at the indicated time points throughout the animal life (FIG. 2). Three groups containing equal proportions of males and females with individual PFIs falling into typical range were separated from the rest of the group at different times of their lives and received short courses of flagellin injected s.c. daily (1 μg/injection) for 5 consecutive days. Control animals received a vehicle (saline) injections. Specific times of injections are indicated in FIG. 2: two groups received one course (44^{th} and 55^{th} week of life), while one group—two courses (18^{th} and 84th weeks of age) of flagellin. PFI was then determined later in life and animals were monitored daily until their death. Functionality of TLR5 signaling in NIH Swiss mice was established in our previous experiments, in which TLR5 agonist (flagellin and its pharmacologicalderivative entolimod) were shown capable of protecting animals from lethal total body irradiation. This experimental design was chosen to reveal the long-lasting effects of treatment with flagellin on mouse biological age determined as PFI. It also allowed us to detect gender-related differences in organismal response to TLR5 agonist.

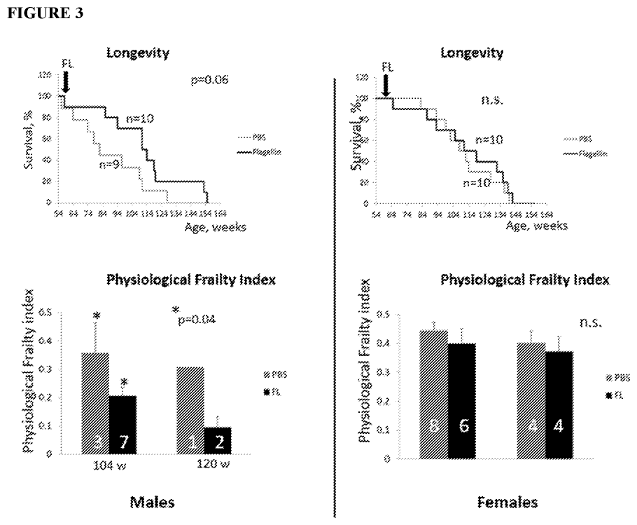

Effect of flagellin treatment on chronological aging in mice. The results of three independent experiments schematically described in FIG. 2 are provided in FIGS. 3-6. All of them demonstrate a substantial slowdown in PFI growth in the groups that received flagellin treatment. This effect was limited to the male mice and was not seen in females. There are reports that estrogen receptors and estradiol modulate TLR5 expression and TLR5—dependent response to flagellin.

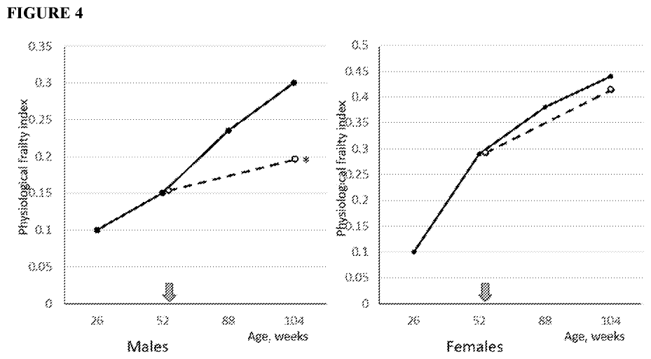

For example, FIG. 3 demonstrates that treatment of NIH Swiss males on the 56th week of age with five consequent daily s.c. injections of 1 μg/mouse of flagellin are translated into a substantially lower PFI nearly one year later—on the 104th week of age. The difference is statistically significant (p=0.04). Graphically, this effect is shown in FIG. 4: at 104 weeks, the average PFI in flagellin treated male group (dashed line) increased by 33% from the time of treatment at 55 weeks, while in control mice it steadily increased by 100% (solid line). Consequently, the group of treated males demonstrated extended longevity vs. vehicle-treated control (average duration of life 22 weeks or 20% longer than control). Neither of the above was observed in female group.

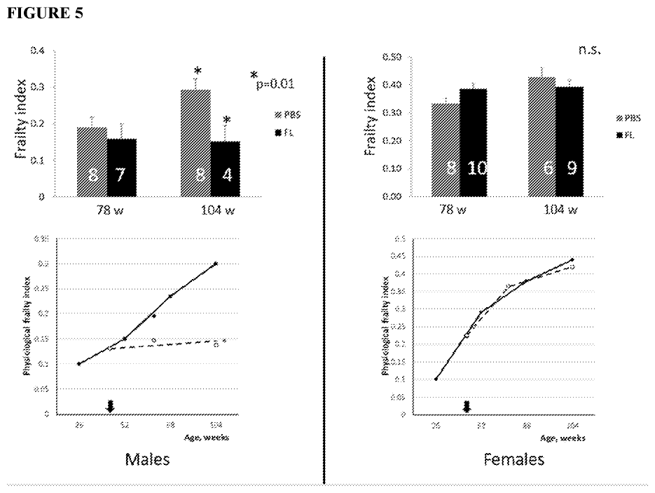

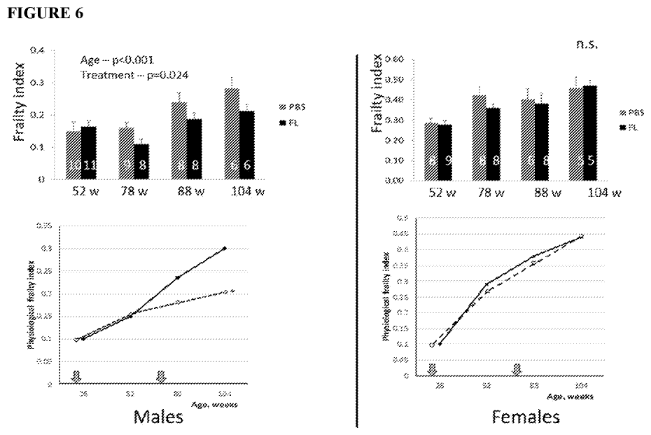

Similar gender-specific effects of flagellin treatment on PFI was observed in Experimental groups 2 and 3 (FIGS. 5, 6). Importantly, the difference between the dynamics of PFI of control and flagellin-treated mice continued to grow within the whole duration of observation suggesting that a single week-long treatment with TLR5 agonist had a long-lasting physiological effect still visible in one-year post-treatment.

### Example 2: Administration of Entolimod to Improve Frailty Index

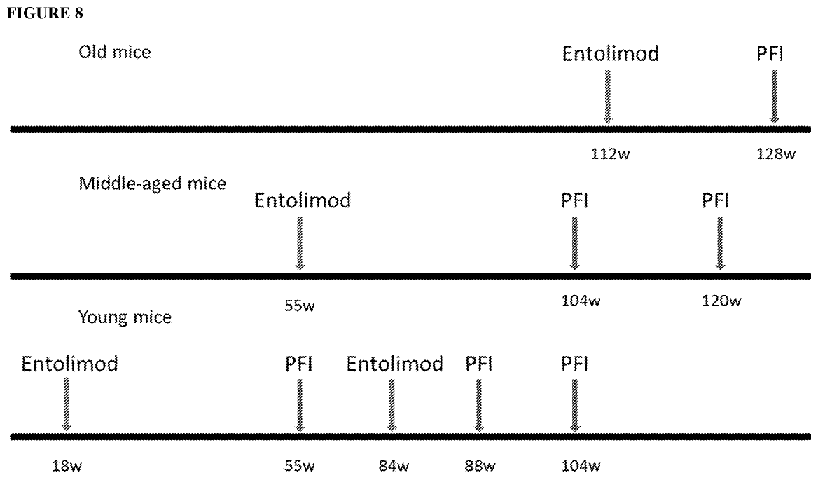

To assess the effect of entolimod treatment on the longevity (lifespan) and overall health (healthspan) of animals, male and female NIH Swiss mice were given 5 daily injections of entolimod (5 μg/mouse, SQ) at different ages: “old” age (112 weeks of age), “middle-age” (55 weeks) and “young” age (18 weeks). Groups treated at “young” age received a second round of treatment at 84 weeks. Control groups consisted of gender- and age-matched mice given SQ injections of PBS instead of entolimod. The experimental design is illustrated in FIG. 8. Mice were housed under standard conditions during the experiment and monitored for mortality and morbidity. Survival was recorded as an indicator of lifespan. In addition, at various times post-treatment (see FIG. 8), tests were performed to determine PFI as a quantitative indicator of healthspan.

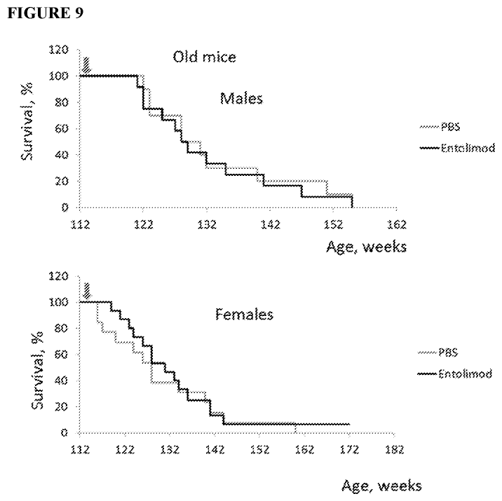

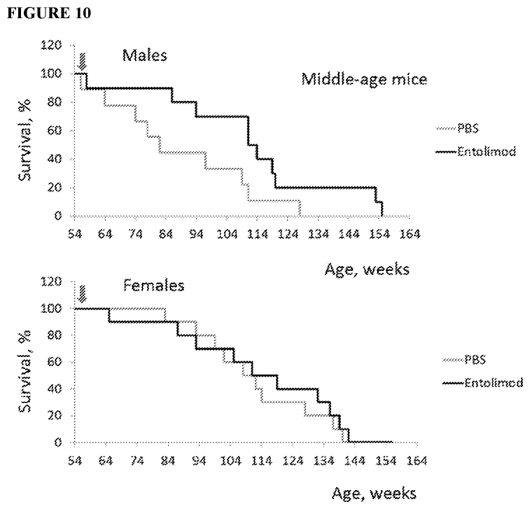

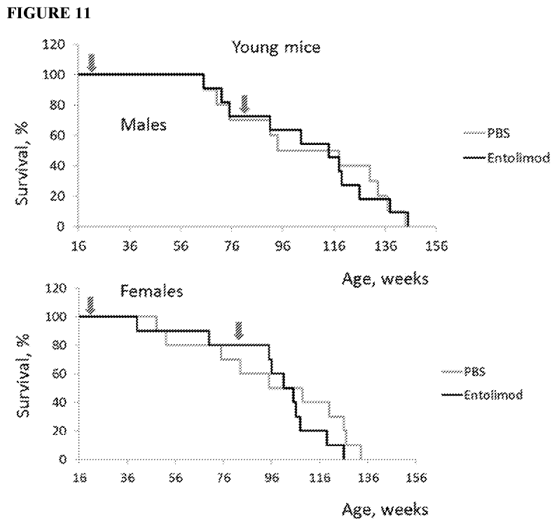

The treated mice were allowed to age naturally under standard housing conditions and their longevity was recorded based on IACUC-approved endpoint criteria for aging animals. FIGS. 9-11 show the impact of treatment with entolimod on lifespan in the age groups tested. Middle-aged male mice showed an increased life span.

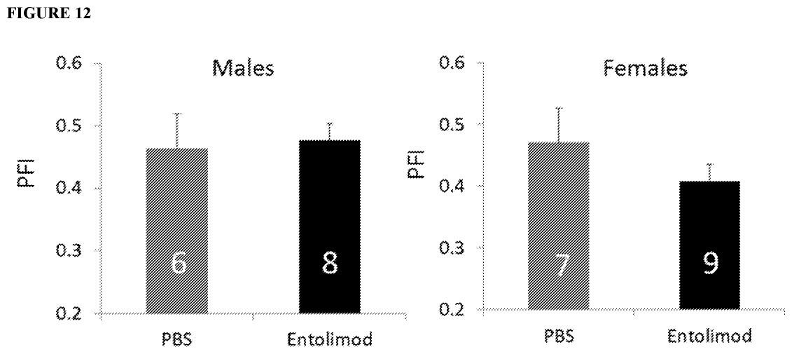

Readouts of the aging process include not only absolute life span, or longevity, but also “health span”, based upon an individual's overall health status. Potential effects of entolimod treatment on health span were evaluated for the treated NIH Swiss mice by determining their Physiological Frailty Index (PFI) at the times post-treatment indicated in FIG. 8. PFI is a quantitative measure based on comparison of physiological parameters between test and reference groups. As shown in FIG. 12, there was no difference in mean PFI values between control and entolimod-treated groups of mice that were treated at “old” age (112 weeks) and evaluated ˜4 months later (at 128 weeks). Similar results were obtained for males and females. These data are consistent with the hypothesis (without wishing to be bound by theory) that treatment of old animals cannot slow down the aging process since the animals have already acquired a number of health deficits.

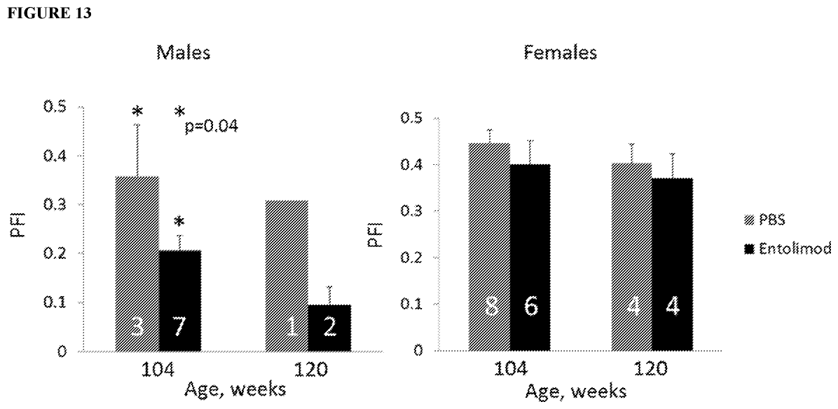

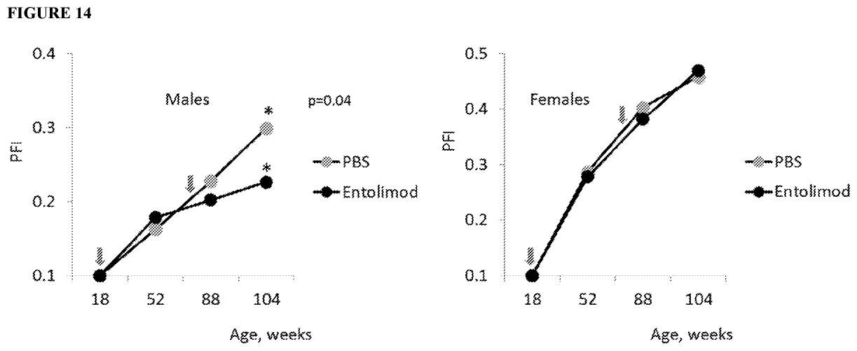

In contrast, treatment of middle-aged (55 week-old) mice with entolimod did have a beneficial effect on aging as measured by PFI at 104 weeks and 120 weeks, albeit only in males (FIG. 13). Mean PFI was significantly lower in entolimod-treated males versus PBS-treated controls at the 104 weeks evaluation timepoint. The same trend was observed at 120 weeks. There was no difference in mean PFI detected in groups of females treated with entolimod versus PBS at 55 weeks of age when evaluated at either 104 or 120 weeks of age (FIG. 13). A similar gender difference was observed in mice treated at a young age (18 weeks) followed by a second treatment at 84 weeks (see FIG. 8 for experimental design). When evaluated at middle-age (55 weeks), there was no difference in mean PFI between entolimod- and PBS-treated groups for either males or females (FIG. 14). However, upon evaluation at 104 weeks, after having received a second treatment during middle-age (at 84 weeks), mean PFI was significantly lower in entolimod-treated males compared to the corresponding PBS-treated group. These mice also showed a noticeable decrease in PFI at 84 weeks. Similar to what was observed for mice treated at 55 weeks of age (FIG. 13), the beneficial effect of entolimod treatment at 18 and 84 weeks was only detected in male mice.

In summary, these results show that a single 5-day course of treatment with entolimod significantly improved health status of mice at older ages, but only when treatment was administered during middle age (in our experiments, between 55 and 84 weeks of age) and only in males.

### Example 3: Administration of Entolimod to a Patient to Improve Frailty Index

A 66-year old male patient is identified that has a recent history of declining frailty index as determined using the Frailty Index (FI), the Physiological Frailty Index (PFI), Fried frailty score, Rockwood frailty index, FRAIL Scale or the modified frailty index. A single cycle that includes one dose of entolimid per day for each of three consecutive days is administered to the patient. The frailty index of the patient is monitored following the entolimid administration using the Frailty Index (FI), the Physiological Frailty Index (PFI), Fried frailty score, Rockwood frailty index, FRAIL Scale or the modified frailty index. The decline in frailty index of the patient is reduced or frailty index is improved following the entolimid administration.

### Example 4: Administration of Entolimod to a Pediatric Patient Who had Received Treatment for Leukemia

A 6 year-old male cancer survivor patient who had previously been treated with chemotherapy for leukemia is identified. One cycle that include one dose of entolimid per day for each of three consecutive days are administered to the patient and a second identical entolimid cycle is administered six months later. The frailty index of the patient is monitored following the entolimid administration and no accelerated aging is observed in the patient.

While the disclosure has been particularly shown and described with reference to specific embodiments, it should be understood by those having skill in the art that various changes in form and detail may be made therein without departing from the spirit and scope of the present disclosure as disclosed herein.

All references referred to herein are incorporated in their entirety. Various embodiments of the present invention may be characterized by the potential claims listed in the paragraphs following this paragraph (and before the actual claims provided at the end of this application). These potential claims form a part of the written description of this application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public.

The embodiments of the invention described above are intended to be merely illustrative; numerous variations and modifications will be apparent to those skilled in the art. All such variations and modifications are intended to be within the scope of the present invention as defined in any appended claims.

As used herein, all headings are simply for organization and are not intended to limit the disclosure in any manner. The content of any individual section may be equally applicable to all sections.

What is claimed is:

1. A method of treating or preventing frailty in a patient, said method comprising
(a) identifying a patient desiring or in need of frailty treatment or prevention, and
(b) administering to said patient a recombinant TLR5 agonist, wherein said TLR5 agonist is not fused to a pathogenic protein antigen.

2. The method of claim 1, wherein frailty comprises an accumulation of deficiencies in major physiological functions, reduction of regeneration capabilities, impaired wound healing and/or increased risk of age-related diseases or disorders.

3. The method of either claim 1 or 2, wherein frailty is measured according to the Physiological Frailty Index.

4. The method of claim 3, wherein the Physiological Frailty Index comprises assessment of one or more parameters selected from grip strength, systolic blood pressure, diastolic blood pressure, blood flow volume, number of blood neutrophils, percentage of blood neutrophils, number of blood monocytes, percentage of blood monocytes, number of lymphocytes, number of red blood cells, hemoglobin levels, hematocrit levels, mean corpuscular volume, mean corpuscular hemoglobin levels, mean corpuscular hemoglobin concentration and keratinocyte-derived cytokine levels.

5. The method of any one of the above claims, wherein the TLR5 agonist is a flagellin or a derivative thereof.

6. The method of claim 5, wherein the TLR5 agonist comprises a polypeptide having an amino acid sequence having at least 95% sequence identity to SEQ ID NO: 1.

7. The method of claim 6, wherein the TLR5 agonist comprises a polypeptide having an amino acid sequence that is SEQ ID NO: 1.

8. The method of claim 5, wherein the TLR5 agonist comprises a polypeptide having an amino acid sequence having at least 95% sequence identity to one of SEQ ID NOs: 2-27.

9. The method of claim 8, wherein the TLR5 agonist comprises a polypeptide having an amino acid sequence of one of SEQ ID NOs: 2-27.

10. The method of any one of the above claims, wherein the patient's Physiological Frailty Index is reduced by about 25% to about 75%.

11. The method of claim 10, wherein the patient's Physiological Frailty Index is reduced by at least about 75%, or about 50%, or about 35%, or about 25%.

12. The method of claim 10, wherein the frailty is associated with aging.

13. The method of any one of the above claims, wherein the patient is middle-aged.

14. The method of claim 13, wherein the patient is between about 36 and about 55 years old.

15. The method of any one of the above claims, wherein the patient is elderly.

16. The method of claim 15, wherein the patient is between about 56 and about 85 years old.

17. The method of any one of the above claims, wherein the biological sex of the patient is male.

18. The method of any one of claims 1-17, wherein the biological sex of the patient is female.

19. A method of treating or preventing an age-related disease or disorder in a patient, said method comprising
(a) identifying a patient desiring or in need of treatment or prevention of an age-related disease or disorder, and
(b) administering to said patient a recombinant TLR5 agonist, wherein said TLR5 agonist is not fused to a pathogenic protein antigen.

20. The method of claim 19, wherein the age-related disease or disorder is characterized by increased cellular senescence.

21. The method of either claim 19 or 20, wherein the age-related disease or disorder is selected from accelerated aging, cardiovascular disease, cerebrovascular disease, peripheral vascular disease, cardiac diastolic dysfunction, benign prostatic hypertrophy, aortic aneurysm, emphysema, atherosclerosis, diabetes, pulmonary fibrosis, blindness, dementia, Alzheimer's disease, kidney dysfunction, osteoarthritis, low grade chronic sterile inflammation, herniated intervertebral disc, frailty, hair loss, hearing loss, vision loss, muscle fatigue, skin conditions, skin nevi, wrinkly skin, hyperpigmentation, scarring, keloid, rosacea, vitiligo, ichthyosis vulgaris, dermatomyositis, actinic keratosis, and sarcopenia.

22. The method of claim 21, wherein the age-related disease or disorder is accelerated aging.

23. The method of claim 22, wherein the accelerate aging is a a Progeroid syndrome, or symptom thereof.

24. The method of claim 23, wherein the Progeroid syndrome is selected from Hutchinson-Gilford progeria syndrome (HGPS), Werner syndrome (WS), Bloom syndrome (BS), Rothmund-Thomson syndrome (RTS), Cockayne syndrome (CS), xeroderma pigmentosum (XP), trichothiodystrophy (TTD), combined xeroderma pigmentosum-Cockayne syndrome (XP-CS), and restrictive dermopathy (RD).

25. The method of claim 22, wherein the accelerated aging is induced by a cancer or a cancer treatment.

26. The method of claim 25, wherein the cancer treatment is selected from one or more of radiotherapy, hormonal therapy, tyrosine kinase inhibitor, anthracycline, alkylating agent, topoisomerase inhibitor, antimetabolites/cytotoxic drug, BRAF inhibitor, antitumor antibiotic, isoquinololine alkaloid, Bcl-2 inhibitor, hematopoietic cell transplantation (HCT), telomerase inhibitor, nucleoside analogue reverse-transcriptase inhibitor, DNA cross-linking agent, ribonucleotide reductase inhibitor, microtubule inhibitor, and miRNA.

27. The method of any one of claims 25-26, wherein the patient was previously afflicted with a cancer.

28. The method of claim 27, wherein the patient is cancer survivor.

29. The method of claim 28, wherein the cancer survivor has completed a cancer treatment and has no apparent evidence of active disease.

30. The method of any one of claims 25-29, wherein the cancer is slectbasal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

31. The method of any one of claims 19-30, wherein the patient is middle-aged.

32. The method of claim 31, wherein the patient is between about 36 and about 55 years old.

33. The method of any one of claims 19-30, wherein the patient is elderly.

34. The method of claim 33, wherein the patient is between about 56 and about 85 years old.

35. The method of any one of claims 25-34, wherein the patient received the cancer treatment before the age of about 18, before the age of about 16, before the age of about 14, before the age of about 12, before the age of about 10, before the age of about 8, before the age of about 6, before the age of about 4, or before the age of about 2.

36. The method of any one of claims 25-35, wherein the TLR5 agonist is administered to the patient for at least one week, or at least one month, or at least six months, or at least one year, or at least two years, or at least three years, or at least four years, or at least five years after the patient received the cancer treatment.

37. The method of any one of claims 25-36, wherein the patient no longer has cancer or the patient is in remission at the time the TLR5 agonist is administered.

38. The method of any one of claims 19-37, wherein administering the TLR5 agonist to the patient treats or prevents the age-related disease or disorder by decreasing cellular senescence.

39. The method of any one of claims 19-38, wherein the TLR5 agonist is flagellin or a derivative thereof.

40. The method of claim 39, wherein the TLR5 agonist comprises a polypeptide having an amino acid sequence having at least 95% sequence identity to SEQ ID NO: 1.

41. The method of claim 40, wherein the TLR5 agonist comprises a polypeptide having an amino acid sequence that is SEQ ID NO: 1.

42. The method of claim 39, wherein the TLR5 agonist comprises a polypeptide having an amino acid sequence having at least 95% sequence identity to one of SEQ ID NOs: 2-27.

43. The method of claim 42, wherein the TLR5 agonist comprises a polypeptide having an amino acid sequence of one of SEQ ID NOs: 2-27.

44. The method of any one of claims 19-43, wherein the biological sex of the patient is male.

45. The method of any one of claims 19-43, wherein the biological sex of the patient is female.

46. The method of any one of the preceding claims, wherein the TLR5 agonist is administered in one or more cycles.

47. The method of claim 46, wherein the cycle involves dosing a patient once per day for one day, once a day for two days, once a day for three days, once a day for four days, or once a day for five days.

48. The method of claim 47, wherein no more than 5 cycles, or no more than 3 cycles, or no more than 2 cycles are administered per year.

49. A TLR5 agonist for use in the treatment or prevention of frailty.

50. Use of a TLR5 agonist in the manufacture of a medicament for treatment or prevention of frailty.

51. A TLR5 agonist for use in the treatment or prevention of an age-related disease or disorder.

52. Use of a TLR5 agonist in the manufacture of a medicament for treatment or prevention of an age-related disease or disorder.


SEQUENCE LISTING (27 sequences)

SEQ ID NO: 1 - protein, 329 aa, Artificial Sequence
  Synthetic polypeptide.
MRGSHHHHHHGMASMTGGQQMGRDLYDDDDKDPMAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNSPGISGGGGGILDSMGTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 2 - protein, 275 aa, Artificial Sequence
  Synthetic polypeptide.
MSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNSPGISGGGGGILDSMGTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLVPRGSHHHHHHG

SEQ ID NO: 3 - protein, 233 aa, Artificial Sequence
  Synthetic polypeptide.
MSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNSPGSTANPLASIDSALSKVDAVRSSLGAIQNRFDSAITNLGNTVTNLNSARSRIEDADYATEVSNMSKAQILQQAGLVPRGSHHHHHHG

SEQ ID NO: 4 - protein, 250 aa, Artificial Sequence
  Synthetic polypeptide.
MSGLRINSAKDDAAGQAIANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILQQAGTSVLAQANQVPQNVLSLLVPRGSHHHHHHG

SEQ ID NO: 5 - protein, 300 aa, Artificial Sequence
  Synthetic polypeptide.
MRGSHHHHHHGMASMTGGQQMGRDLYDLVPRGSAKDPSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNSPGISGGGGGILDSMGTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 6 - protein, 250 aa, Artificial Sequence
  Synthetic polypeptide.
MSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNSPGSTANPLASIDSALSKVDAVRSSLGAIQNRFDSAITNLGNTVTNLNSARSRIEDADYATEVSNMSKAQILQQAGTSVLAQANQVPQNVLSLLVPRGSHHHHHHG

SEQ ID NO: 7 - protein, 293 aa, Artificial Sequence
  Synthetic polypeptide.
MGHHHHHHSGMEEFNMRINTNVAAMNTYSRLTAANTAKSNSLAKLSSGLRINKAGDDAAGLAISEKMKSQIGGLTQAKRNAQDGISLVQTAEGALNETHSILERMRDLAVQGSNGTLTSSDRGSINKELKALHQELTRISNTTEFNTQKLFSQTKQKSVTFTFQIGANAGQTLSVAITAMSGEALLVSTDAKFSLNAAGTNAGAMIKSIDAAIAKVSDQRADLGAVQNRLEHTINNLTATNENLSDANSRIRDVDMAEEMMTFTKSNILSQAATSMLAQANAMPNSVLNLLQG

SEQ ID NO: 8 - protein, 280 aa, Artificial Sequence
  Synthetic polypeptide.
MGHHHHHHSGMRINHNISALNAWRNIDQTQYSMSKTLERLSSGLRINRAGDDAAGLAISEKMRGQIKGLNMAIKNAQDAISLIQTAEGALTEVHSILQRMRELAVQAASDTNTNVDREQIQKEIDQLREEIDRIARTTEFNTKKLLDGKLEGFRSQVDAKVVTGGNINVQLGTVSSKAVEGTYVIEVGAAERAIMVVDAAIHRVSTARAALGAIQNRLEHTISNLGVAAENLTAAESRIRDADMAKEMMEFTKQQILLQSSMAMLAQSNTLPQNVLQLMR

SEQ ID NO: 9 - protein, 168 aa, Artificial Sequence
  Synthetic polypeptide.
MGHHHHHHSGLNMAIKNAQDAISLIQTAEGALTEVHSILQRMRELAVQAASDTNTNVDREQIQKEIDQLREEIDRIARTTEFNTKKLLDGKLEGFRSQVDAKVVTGGNINVQLGTVSSKAVEGTYVIEVGAAERAIMVVDAAIHRVSTARAALGAIQNRLEHTISNLG

SEQ ID NO: 10 - protein, 285 aa, Artificial Sequence
  Synthetic polypeptide.
MGHHHHHHSGMSLRINNNIEALNAWRALNSTSNALQKSMEKLSSGLRINRAGDDAAGLAISEKLRAQIRGLNQAIRNAQDGISLIQTAEGGLSEIQNILQRMRELGVQAANGTLNNQDISAITTELNQLFNEIDRIAGATEFNTKNLLAVSTGLVVTLQVGANAGQVIAFTIDNAGTASLGLSSADLAINDNASASAFISKVDSALQKVSTYRANLGSIQNRLEHTIANLGIASENLSASESRIRDVDMAAEMMNFTKNQILQQAGVAILAQANQAPQAVLQLLR

SEQ ID NO: 11 - protein, 171 aa, Artificial Sequence
  Synthetic polypeptide.
MGHHHHHHSGLNQAIRNAQDGISLIQTAEGGLSEIQNILQRMRELGVQAANGTLNNQDISAITTELNQLFNEIDRIAGATEFNTKNLLAVSTGLVVTLQVGANAGQVIAFTIDNAGTASLGLSSADLAINDNASASAFISKVDSALQKVSTYRANLGSIQNRLEHTIANLG

SEQ ID NO: 12 - protein, 146 aa, Artificial Sequence
  Synthetic polypeptide.
MGHHHHHHSGLNQAIRNAQDGISLIQTAEGGLSEIQNILQRMRELGVQAANGTLNNQDISAITTELNQLFNEIDRIAGATEFNTKNLLAAGTASLGLSSADLAINDNASASAFISKVDSALQKVSTYRANLGSIQNRLEHTIANLG

SEQ ID NO: 13 - protein, 116 aa, Artificial Sequence
  Synthetic polypeptide.
MGHHHHHHSASAFISKVDSALQKVSTYRANLGSIQNRLEHTIANLGPDGLNQAIRNAQDGISLIQTAEGGLSEIQNILQRMRELGVQAANGTLNNQDISAITTELNQLFNEIDRIA

SEQ ID NO: 14 - protein, 103 aa, Artificial Sequence
  Synthetic polypeptide.
MGHHHHHHSNNQDISAITTELNQLFNEIDRIAGATGSGGLSEIQNILQRMRELGVQAANGTLNGGSASAFISKVDSALQKVSTYRANLGSIQNRLEHTIANLG

SEQ ID NO: 15 - protein, 170 aa, Artificial Sequence
  Synthetic polypeptide.
MGHHHHHHSGLAQASRNAQDAISIAQTAEGALDETQSILQRVRELGVQGANGTLTADDINALQAEVDQLIAEIDRIAGATEFNTQNLLDGSFTTKAFQVGANSGQNMTLTIGKMDTTTLGLSSADLAINDNAFANGAISTVDSALQKVSAERAKLGAIQNRLEHTIANLG

SEQ ID NO: 16 - protein, 170 aa, Artificial Sequence
  Synthetic polypeptide.
MGHHHHHHSGLAQASRQAQDAISIAQTAEGALDETQSILQRVRELGVQGADGTLTADDIDALQAEVDQLIAEIDRIAGATEFATQKLLDGSFTTKAFQVGAASGQDVTLTIGKVDTTTLGLSSADLAIDSAAFADGAISTVDSALQKVSAERAKLGAIQNRLEHTIAQLG

SEQ ID NO: 17 - protein, 274 aa, Artificial Sequence
  Synthetic polypeptide.
MRGSHHHHHHGMASMTGGQQMGRDLYDDDDKDPMAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNSPGISGGGGGILDSMGTLINEDAAAAKKSTANPLASIDSALSKVDAVRSSLGAIQNRFDSAITNL

SEQ ID NO: 18 - protein, 276 aa, Artificial Sequence
  Synthetic polypeptide.
MRGSHHHHHHGMASMTGGQQMGRDLYDDDDKDPFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNSPGISGGGGGILDSMGTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 19 - protein, 323 aa, Artificial Sequence
  Synthetic polypeptide.
MRGSHHHHHHGMASMTGGQQMGRDLYDDDDKDPMAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLIPGISGGGGGILDSMGTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 20 - protein, 270 aa, Artificial Sequence
  Synthetic polypeptide.
MRGSHHHHHHGMASMTGGQQMGRDLYDDDDKDPFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLIPGISGGGGGILDSMGTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 21 - protein, 316 aa, Artificial Sequence
  Synthetic polypeptide.
MRGSHHHHHHGMASMTGGQQMGRDLYDDDDKDPMAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIIPGISGGGGGILDSMGTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 22 - protein, 263 aa, Artificial Sequence
  Synthetic polypeptide.
MRGSHHHHHHGMASMTGGQQMGRDLYDDDDKDPFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIIPGISGGGGGILDSMGTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 23 - protein, 268 aa, Artificial Sequence
  Synthetic polypeptide.
MRGSHHHHHHGMASMTGGQQMGRDLYDDDDKDPMAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLIPGISGGGGGILDSMGTLINEDAAAAKKSTANPLASIDSALSKVDAVRSSLGAIQNRFDSAITNL

SEQ ID NO: 24 - protein, 261 aa, Artificial Sequence
  Synthetic polypeptide.
MRGSHHHHHHGMASMTGGQQMGRDLYDDDDKDPMAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIIPGISGGGGGILDSMGTLINEDAAAAKKSTANPLASIDSALSKVDAVRSSLGAIQNRFDSAITNL

SEQ ID NO: 25 - protein, 282 aa, Artificial Sequence
  Synthetic polypeptide.
MRGSHHHHHHGMASMTGGQQMGRDLYDDDDKDPMAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQIPGISGGGGGILDSMGTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 26 - protein, 229 aa, Artificial Sequence
  Synthetic polypeptide.
MRGSHHHHHHGMASMTGGQQMGRDLYDDDDKDPFTSNIKGLTQASRNANDGISIAQTTEGALNEINNNLQRVRELSVQATNGTNSDSDLKSIQDEIQQRLEEIDRVSNQIPGISGGGGGILDSMGTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 27 - protein, 505 aa, Artificial Sequence
  Synthetic polypeptide.
MAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNGPKEATVGDLKSSFKNVTGYDTYAAGADKYRVDINSGAVVTDAAAPDKVYVNAANGQLTTDDAENNTAVDLFKTTKSTAGTAEAKAIAGAIKGGKEGDTFDYKGVTFTIDTKTGDDGNGKVSTTINGEKVTLTVADIATGAADVNAATLQSSKNVYTSVVNGQFTFDDKTKNESAKLSDLEANNAVKGESKITVNGAEYTANATGDKITLAGKTMFIDKTASGVSTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR